(19) United States
(12) Patent Application Publication
(10) Pub. No.: US 2022/0009967 A1
(43) Pub. Date: Jan. 13, 2022

(54) Isolation and Semi-Synthesis of Rufomycin Analogs

(71) Applicant: THE BOARD OF TRUSTEES OF THE UNIVERSITY OF ILLINOIS, Urbana, IL (US)

(72) Inventor: Scott G. Franzblau, Chicago, IL (US)

(73) Assignee: THE BOARD OF TRUSTEES OF THE UNIVERSITY OF ILLINOIS, Urbana, IL (US)

(21) Appl. No.: 17/305,713

(22) Filed: Jul. 13, 2021

Related U.S. Application Data
(60) Provisional application No. 63/050,902, filed on Jul. 13, 2020.

(51) Int. Cl. C07K 7/64; A61K 38/00

(52) U.S. Cl. CPC C07K 7/64; A61K 38/00

(57) ABSTRACT

Provided herein are compounds having a structure according to formula I, pharmaceutical compositions thereof, and methods of treating a bacterial infection thereof.

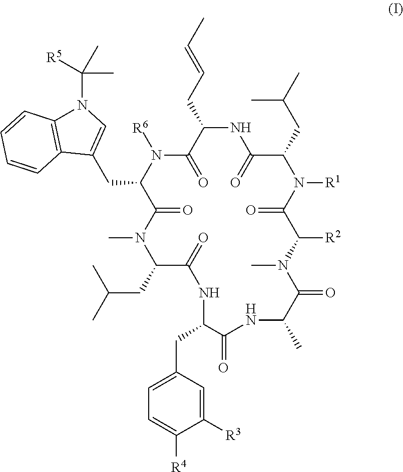

### CROSS-REFERENCE TO RELATED APPLICATION

This application claims the benefit of U.S. Provisional Patent Application No. 63/050,902, filed Jul. 13, 2020, which is incorporated by reference herein in its entirety

### STATEMENT OF US GOVERNMENT SUPPORT

This invention was made with government support under Grant Number U19A1142735, awarded by the National Institutes of Health. The government has certain rights in the invention.

### BACKGROUND

Rufomycin, per se, is a cyclic peptide produced by Streptomyces stratus with potent in vitro growth inhibitory activity against Mycobacterium tuberculosis resulting in a perturbation of protein degradation. Rufomycin analogues comprise a family of cyclic heptapeptides, which contain leucine (Leu), two N-methylleucines (NMeLeu), alanine (Ala), and three non-proteinogenic amino acids, N-dimethylallyl(epoxy)tryptophan, trans-2-crotylglycine (TrcGly), and m-nitrotyrosine (mNO₂Try). There is currently a need for rufomycin analogues that exhibit better in vitro potency compared to the existing anti-tuberculosis drugs and that exhibit activity against drug resistant tuberculosis.

### SUMMARY

Provided herein are compounds having a structure of formula I or a pharmaceutically acceptable salt thereof

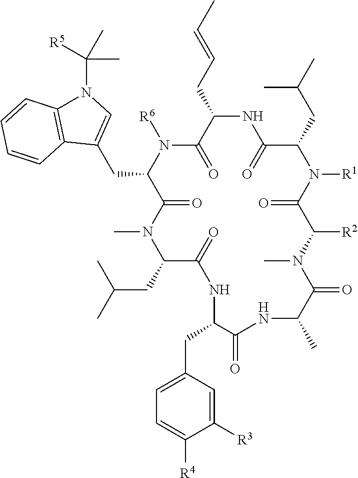

wherein,
R¹ and R², together, are selected from

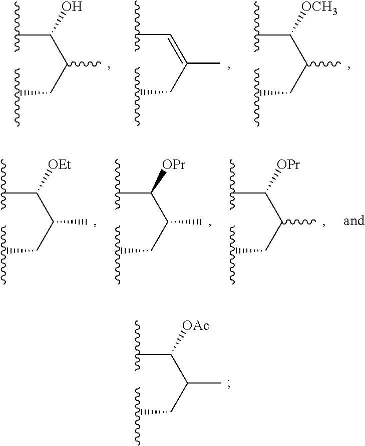

R³ and R⁴ are independently selected from NO₂, OH, OAc, OCH₃, OBz, NH₂, and

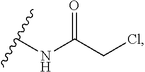 [SMILES: *NC(=O)CCl]

or R³ and R⁴ together with the carbon atoms to which they are attached, form a five- to seven-membered substituted or unsubstituted heterocycloalkyl comprising 1 to 3 ring heteroatoms selected from O and N; R⁵ is selected from

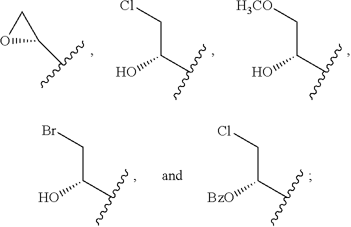

and, R⁶ is selected from H and C₁-C₃ alkyl; provided when R¹ and R² together are

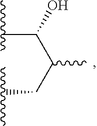

R³ is NO₂, R⁴ is OH, and R⁵ is

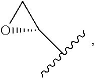 [SMILES: *[C@H]1CO1]

that R⁶ is not H; when R¹ and R² together are

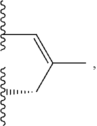

R³ is NO₂, R⁴ is OH, and R⁵ is

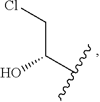 [SMILES: *[C@H](O)CCl]

that R⁶ is not H; when R¹ and R² together are

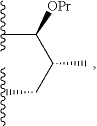

R³ is NO₂, R⁴ is OH, and R⁵ is

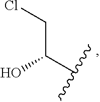 [SMILES: *[C@H](O)CCl]

that R⁶ is not H; and when R¹ and R² together are

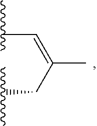

R³ is NO₂, R⁴ is OH, and R⁵ is

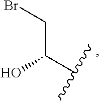 [SMILES: *[C@H](O)CBr]

that R⁶ is not H.

Also provided herein are compounds or a pharmaceutically acceptable salt thereof having a structure selected from the group of

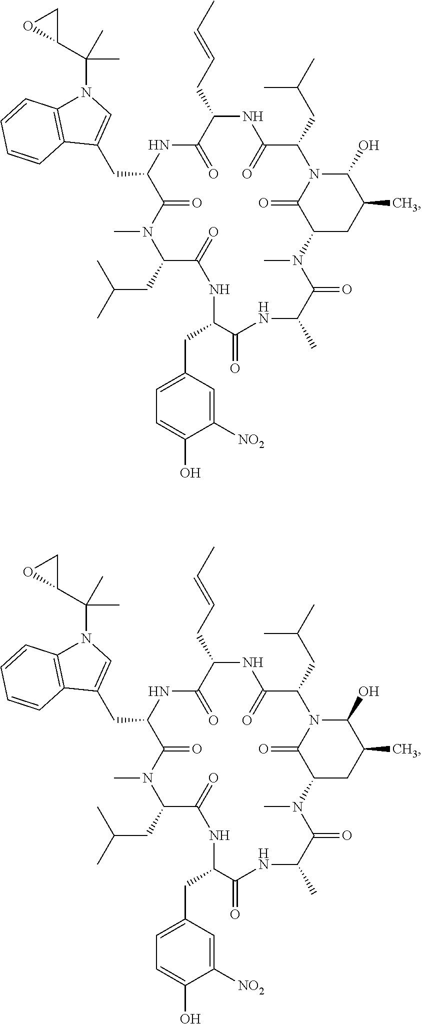 [SMILES: C/C=C/C[C@@H]1NC(=O)[C@H](CC(C)C)N2C(=O)[C@H](C[C@H](C)[C@H]2O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O ; C/C=C/C[C@@H]1NC(=O)[C@H](CC(C)C)N2C(=O)[C@H](C[C@H](C)[C@@H]2O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O]

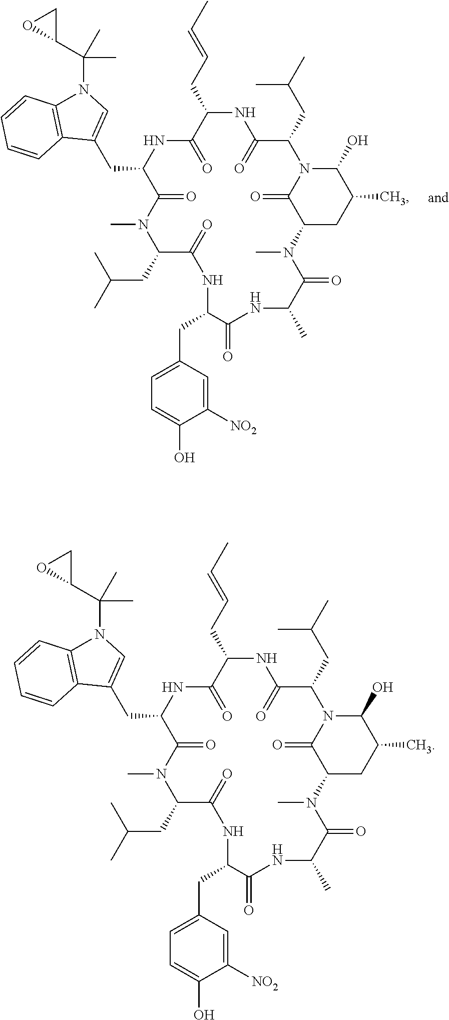 [SMILES: C/C=C/C[C@@H]1NC(=O)[C@H](CC(C)C)N2C(=O)[C@H](C[C@@H](C)[C@H]2O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O ; C/C=C/C[C@@H]1NC(=O)[C@H](CC(C)C)N2C(=O)[C@H](C[C@@H](C)[C@@H]2O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O]

Also provided herein are methods of treating a bacterial infection, comprising administering to a subject in need thereof a therapeutically effective amount of a compound of formula I, or a pharmaceutically acceptable salt thereof.

Also provided herein are pharmaceutical compositions comprising a therapeutically effective amount of the compound of formula I, or a pharmaceutically acceptable salt thereof, and an excipient.

Also provided herein are methods of treating a bacterial infection, comprising administering to a subject in need thereof a therapeutically effective amount of a compound having a structure selected from the group of

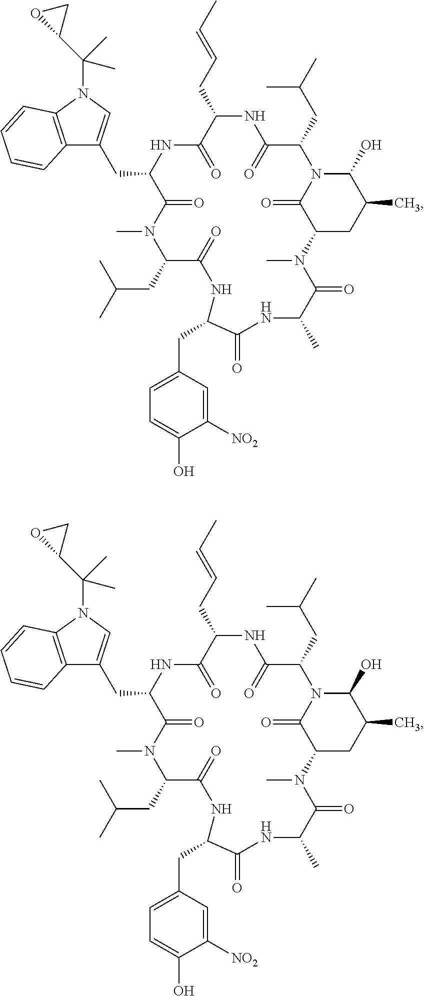 [SMILES: C/C=C/C[C@@H]1NC(=O)[C@H](CC(C)C)N2C(=O)[C@H](C[C@H](C)[C@H]2O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O ; C/C=C/C[C@@H]1NC(=O)[C@H](CC(C)C)N2C(=O)[C@H](C[C@H](C)[C@@H]2O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O]

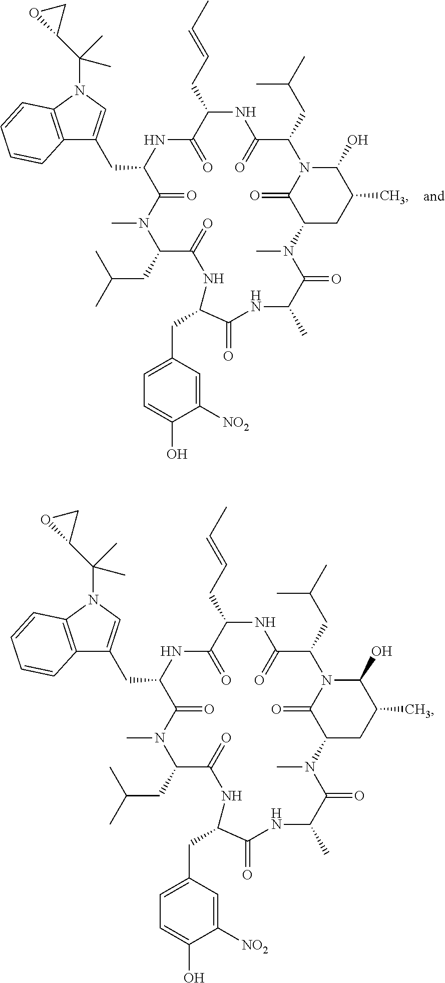 [SMILES: C/C=C/C[C@@H]1NC(=O)[C@H](CC(C)C)N2C(=O)[C@H](C[C@@H](C)[C@H]2O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O ; C/C=C/C[C@@H]1NC(=O)[C@H](CC(C)C)N2C(=O)[C@H](C[C@@H](C)[C@@H]2O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O]

or a pharmaceutically acceptable salt thereof.

### BRIEF DESCRIPTION OF FIGURES

FIG. 1 shows a graph of the binding affinity of the compounds of the disclosure using the equilibrium dissociation constants (KD) to caseinolytic protein C1 (ClpC1) N-terminal domain (NTD) (nM) versus ClpC1 full-length protein (FL) (nM).

FIG. 2 shows a graph of the binding affinity of the compounds of the disclosure with ClpC1-NTD (nM) and the minimum inhibitory concentration (MIC) (nM) for the compounds of the disclosure against Mycobacterium tuberculosis.

### DETAILED DESCRIPTION

Provided herein are compounds having a structure according to formula I or a pharmaceutically acceptable salts thereof. The compounds can provide benefits, including higher potency and activity as compared to previously reported ruformycin and analogues thereof (Ma et al., Nature Comm. 2017, 8, 391; Choules et al., Antimicrob. Agents Chemother. 2019, 63, e02204-02218; Takita et al., J. Antibiot. Ser. A 1965, 18, 135-136; Zhou et al., J. Nat. Prod. 2020, 83, 657-667). Also provided herein are pharmaceutical compositions comprising a therapeutically effective amount of the compound of formula I, or a pharmaceutically acceptable salt thereof, and an excipient. Further, provided herein are methods of treating a bacterial infection, comprising administering to a subject in need thereof a therapeutically effective amount of the compound of formula I, or a pharmaceutically acceptable salt thereof.

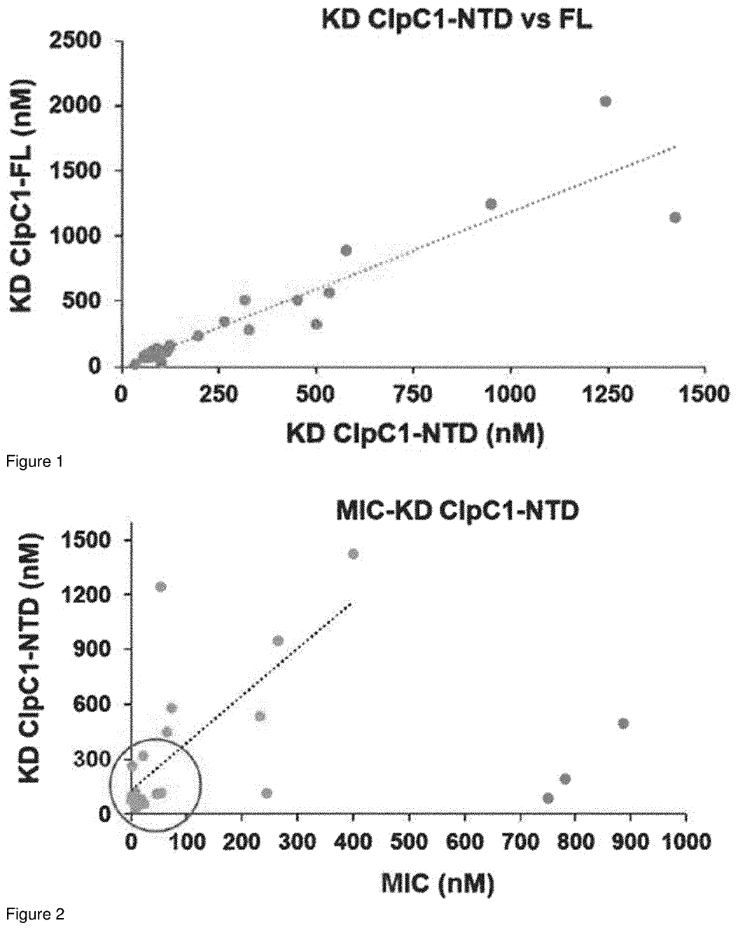

As further described below, the compounds of formula I or a pharmaceutically acceptable salt thereof can have several advantages, for example, the compounds disclosed herein have low minimum inhibitory concentration (MIC) against Mycobacterium tuberculosis and rifampicin resistant Mycobacterium tuberculosis, as well as showing high binding affinities with ClpC1-NTD (FIGS. 1 and 2). Further, the compounds as provided herein, e.g., compounds of formula I, are advantageous as they represent a valuable addition to rufomycin analogues in the art.

The methods of treating a bacterial infection of the disclosure are useful for treating these conditions in that they can inhibit the onset, growth, or spread of the condition, cause regression of the condition, cure the condition, or otherwise improve the general well-being of a subject afflicted with, or at risk of, contracting the condition. Thus, in accordance with the presently disclosed subject matter, the terms “treat”, “treating”, and grammatical variations thereof, as well as the phrase “method of treating”, are meant to encompass one or more desired therapeutic interventions, including but not limited to a method for treating an existing infection in a subject. Compounds of the disclosure can also be useful in methods for the prophylaxis (i.e., preventing) of infection, such as in a subject that has been exposed to a microbe as disclosed herein or that has an expectation of being exposed to a microbe as disclosed herein.

The use of the terms “a,” “an,” “the,” and similar referents in the context of the disclosure herein (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure herein and is not a limitation on the scope of the disclosure herein unless otherwise indicated. No language in the specification should be construed as indicating any non-claimed element as essential to the practice of the disclosure herein.

The term “alkyl” used herein refers to a saturated or unsaturated straight or branched chain hydrocarbon group of one to three carbon atoms, including, but not limited to, methyl, ethyl, n-propyl, and isopropyl. Alkyl groups optionally can be substituted, for example, with hydroxy (OH), halide, thiol (SH), aryl, heteroaryl, cycloalkyl, heterocycloalkyl, and amino. It is specifically contemplated that in the compounds described herein the alkyl group consists of 1-3 carbon atoms.

The term “heterocycloalkyl” used herein refers to a hydrocarbon group arranged in a ring, wherein 1 to 3 of the carbon ring atoms are replaced with O and/or N. The heterocycloalkyl group can be substituted with one or more substituents, such as alkyl, halo, OH, SH, amino, substituted amino, carboxy, aryl, or heteroaryl. Nonlimiting examples of heterocycloalkyl groups include piperidine, tetrahydrofuran, tetrahydropyran, dihydrofuran, morpholine, and the like. The heterocycloalkyl groups described herein can be isolated or fused to another heterocycloalkyl group, a cycloalkyl group, an aryl group, and/or a heteroaryl group. In some embodiments, the heterocycloalkyl groups described herein comprise one oxygen ring atom (e.g., oxiranyl, oxetanyl, tetrahydrofuranyl, and tetrahydropyranyl).

In some cases, the substituent group(s) is (are) one or more group(s) individually and independently selected from alkyl, cycloalkyl, aryl, heterocyclyl, heteroaryl, hydroxy, alkoxy, aryloxy, mercapto, alkylthio, arylthio, cyano, halo, carbonyl, thiocarbonyl, alkoxycarbonyl, nitro, silyl, trihalomethanesulfonyl, trifluoromethyl, and amino, including mono- and di-substituted amino groups, and the protected derivatives thereof.

Asymmetric carbon atoms can be present. All such isomers, including diastereomers and enantiomers, as well as the mixtures thereof, are intended to be included in the scope of the disclosure herein. In certain cases, compounds can exist in tautomeric forms. All tautomeric forms are intended to be included in the scope of the disclosure herein. Likewise, when compounds contain an alkenyl or alkenylene group, there exists the possibility of cis- and trans-isomeric forms of the compounds. Both cis- and trans-isomers, as well as the mixtures of cis- and trans-isomers, are contemplated.

Compounds of the Disclosure

Provided herein are a compound having a structure according to Formula I or a pharmaceutically acceptable salt thereof:

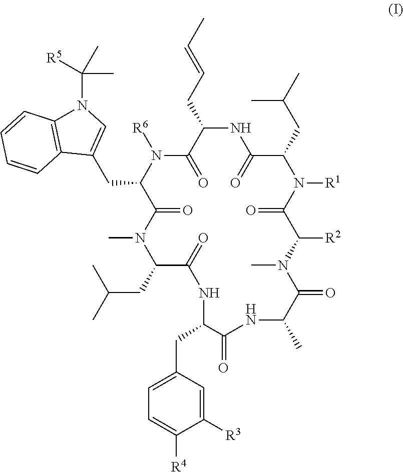

wherein,

R¹ and R², together are selected from

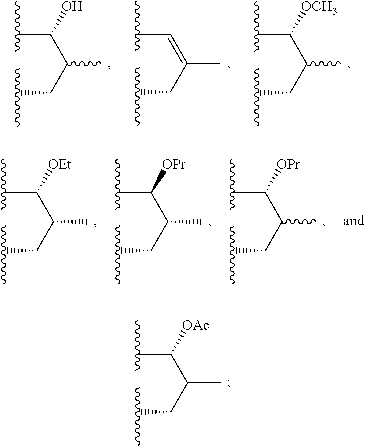

R³ and R⁴ are independently selected from NO₂, OH, OAc, OCH₃, OBz, NH₂, and

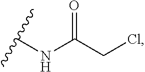 [SMILES: *NC(=O)CCl]

or R³ and R⁴ together with the carbon atoms to which they are attached, form a five- to seven-membered substituted or unsubstituted heterocycloalkyl comprising 1 to 3 ring heteroatoms selected from 0 and N;

R⁵ is selected from

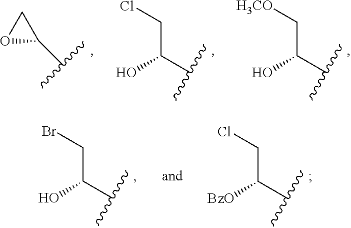

R⁶ is selected from H and C₁-C₃ alkyl;

provided when R¹ and R² together are

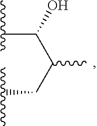

R³ is NO₂, R⁴ is OH, and R⁵ is

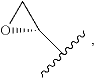 [SMILES: *[C@H]1CO1]

that R⁶ is not H; when R¹ and R² together are

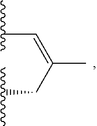

R³ is NO₂, R⁴ is OH, and R⁵ is

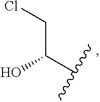 [SMILES: *[C@H](O)CCl]

that R⁶ is not H; when R¹ and R² together are

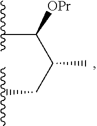

R³ is NO₂, R⁴ is OH, and R⁵ is

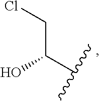 [SMILES: *[C@H](O)CCl]

that R⁶ is not H; and when R¹ and R² together are

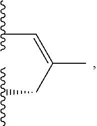

R³ is NO₂, R⁴ is OH, and R⁵ is

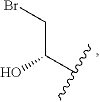 [SMILES: *[C@H](O)CBr]

that R⁶ is not H.

In general, R¹ and R², together are selected from

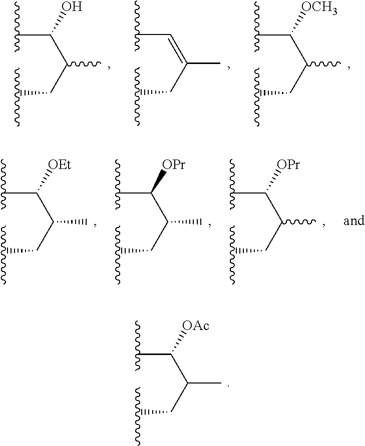

In embodiments, R¹ and R² together are selected from

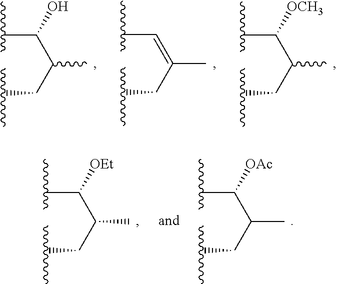

In embodiments, R¹ and R² together are selected from

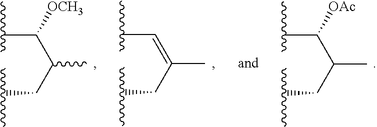

In embodiments, R¹ and R² together are selected from

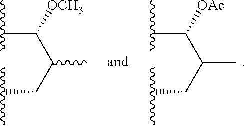

In embodiments, R¹ and R², together are selected from

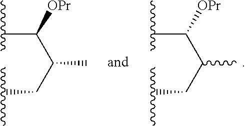

In embodiments, R¹ and R², together are selected from

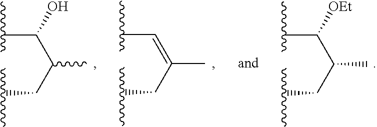

In general, R³ and R⁴ are independently selected from NO₂, OH, OAc, OCH₃, OBz, NH₂, and

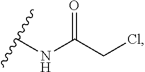 [SMILES: *NC(=O)CCl]

or R³ and R⁴ together with the carbon atoms to which they are attached, form a five- to seven-membered substituted or unsubstituted heterocycloalkyl comprising 1 to 3 ring heteroatoms selected from 0 and N. In embodiments, R³ is NO₂, NH₂, or

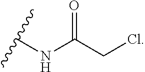 [SMILES: *NC(=O)CCl]

In embodiments, R³ is

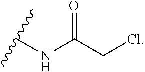 [SMILES: *NC(=O)CCl]

In embodiments, R³ is NO₂. In embodiments, R⁴ is NH₂. In embodiments, R⁴ is OH, OAc, OCH₃, or OBz. In embodiments, R⁴ is OH, OAc, or OCH₃. In embodiments, R⁴ is OH. In embodiments, R⁴ is OAc. In embodiments, R⁴ is OCH₃. In embodiments, R³ and R⁴ together with the carbon atoms to which they are attached, form a five- to seven-membered substituted or unsubstituted heterocycloalkyl comprising 1 to 3 ring heteroatoms selected from O and N. In embodiments, R³ and R⁴ together with the carbon atoms to which they are attached, form a five-membered substituted or unsubstituted heterocycloalkyl comprising 1 to 3 ring heteroatoms selected from O and N. In embodiments, R³ and R⁴ together with the carbon atoms to which they are attached, form a seven-membered substituted or unsubstituted heterocycloalkyl comprising 1 to 3 ring heteroatoms selected from O and N. In embodiments, R³ and R⁴ together with the carbon atoms to which they are attached, form a five-membered substituted or unsubstituted heterocycloalkyl comprising 2 ring heteroatoms selected from O and N. In embodiments, R³ and R⁴ together with the carbon atoms to which they are attached, form a seven-membered substituted or unsubstituted heterocycloalkyl comprising 2 ring heteroatoms selected from O and N. In embodiments, R³ and R⁴ together are selected from

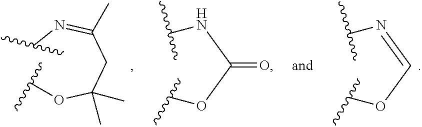

As used herein, the term “Ac” is an acetyl group. As used herein, the term “Bz” is a benzoyl group.

In general, R⁵ is selected from

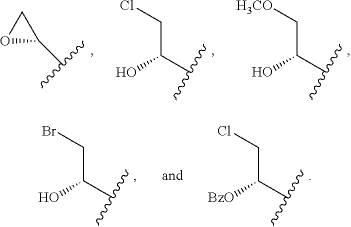

In embodiments, R⁵ is selected from

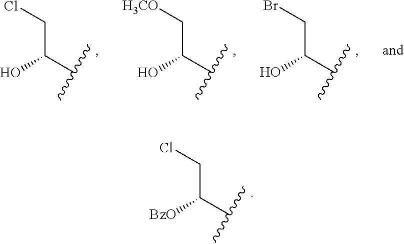

In embodiments, R⁵ is selected from

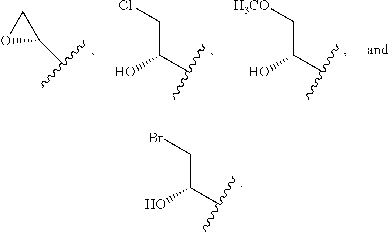

In embodiments, R⁵ is

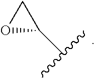 [SMILES: *[C@H]1CO1]

In embodiments, R⁵ is

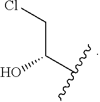 [SMILES: *[C@H](O)CCl]

In embodiments, R⁵ is

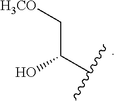 [SMILES: *[C@H](O)COC]

In embodiments, R⁵ is

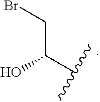 [SMILES: *[C@H](O)CBr]

In embodiments, R⁵ is

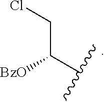

In general, R⁶ is selected from H and C₁-C₃ alkyl. In embodiments, R⁶ is H. In embodiments, R⁶ is C₁-C₃ alkyl. In embodiments, R⁶ is selected from methyl, ethyl, isopropyl, and n-propyl. In embodiments, R⁶ is methyl. In embodiments, R⁶ is ethyl. In embodiments, R⁶ is isopropyl or n-propyl.

In embodiments, the compound of formula I or a pharmaceutically acceptable salt thereof is selected from

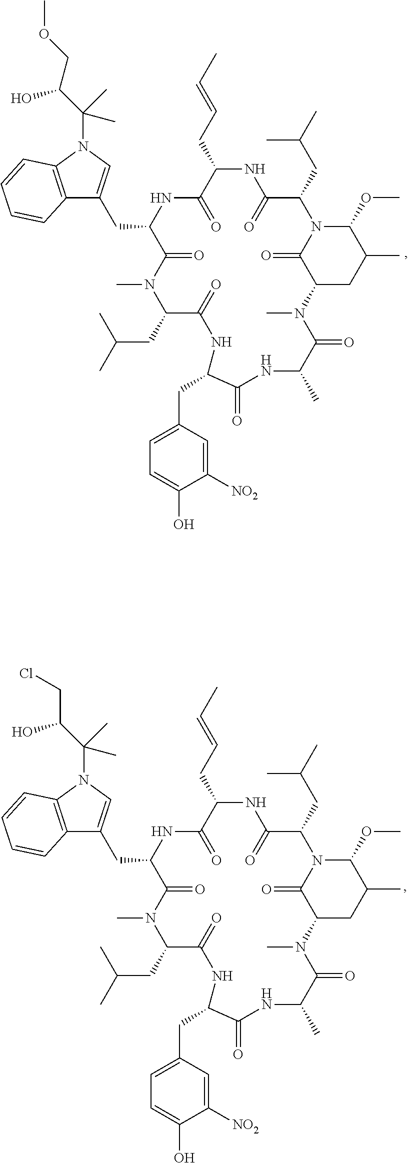 [SMILES: C/C=C/C[C@@H]1NC(=O)[C@H](CC(C)C)N2C(=O)[C@H](CC(C)[C@H]2OC)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H](O)COC)c3ccccc23)NC1=O ; C/C=C/C[C@@H]1NC(=O)[C@H](CC(C)C)N2C(=O)[C@H](CC(C)[C@H]2OC)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H](O)CCl)c3ccccc23)NC1=O]

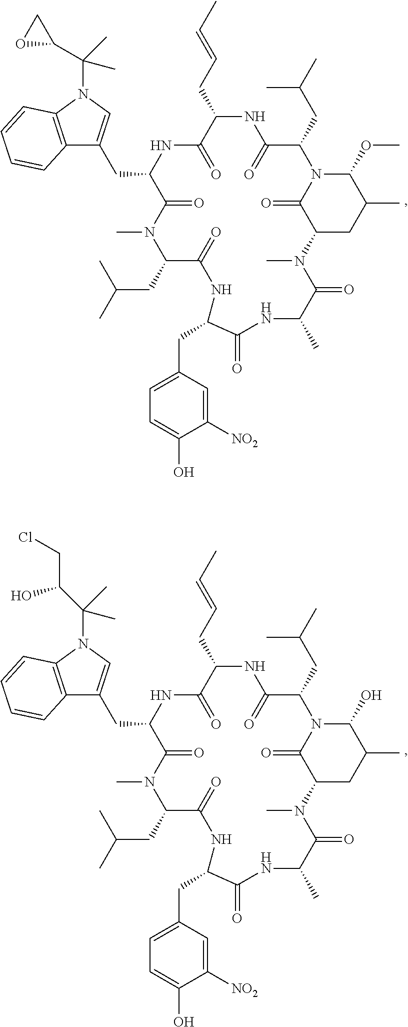 [SMILES: C/C=C/C[C@@H]1NC(=O)[C@H](CC(C)C)N2C(=O)[C@H](CC(C)[C@H]2OC)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O ; C/C=C/C[C@@H]1NC(=O)[C@H](CC(C)C)N2C(=O)[C@H](CC(C)[C@H]2O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H](O)CCl)c3ccccc23)NC1=O]

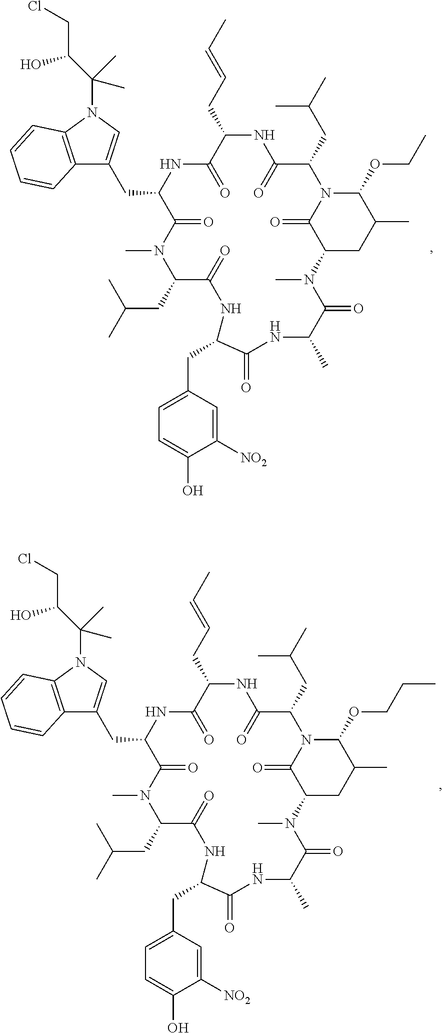 [SMILES: C/C=C/C[C@@H]1NC(=O)[C@H](CC(C)C)N2C(=O)[C@H](CC(C)[C@H]2OCC)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H](O)CCl)c3ccccc23)NC1=O ; C/C=C/C[C@@H]1NC(=O)[C@H](CC(C)C)N2C(=O)[C@H](CC(C)[C@H]2OCCC)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H](O)CCl)c3ccccc23)NC1=O]

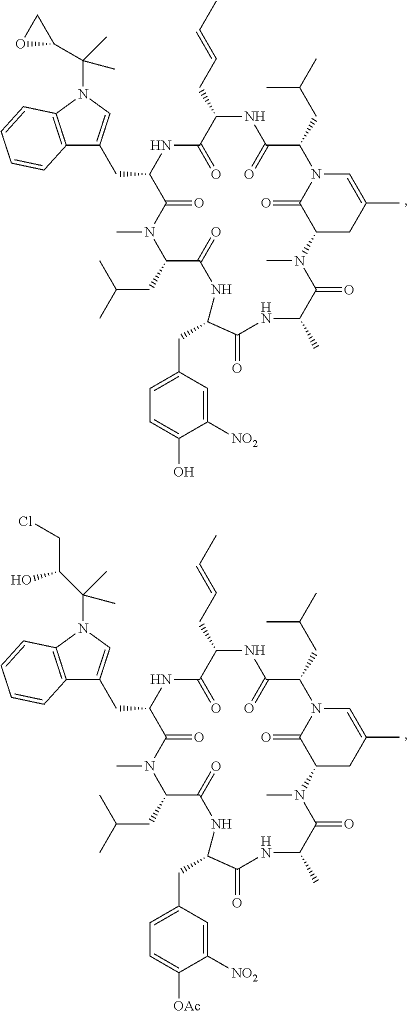 [SMILES: C/C=C/C[C@@H]1NC(=O)[C@H](CC(C)C)N2C=C(C)C[C@@H](C2=O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O ; C/C=C/C[C@@H]1NC(=O)[C@H](CC(C)C)N2C=C(C)C[C@@H](C2=O)N(C)C(=O)[C@H](C)NC(=O)[C@H](Cc2ccc(OC(C)=O)c([N+](=O)[O-])c2)NC(=O)[C@H](CC(C)C)N(C)C(=O)[C@H](Cc2cn(C(C)(C)[C@H](O)CCl)c3ccccc23)NC1=O]

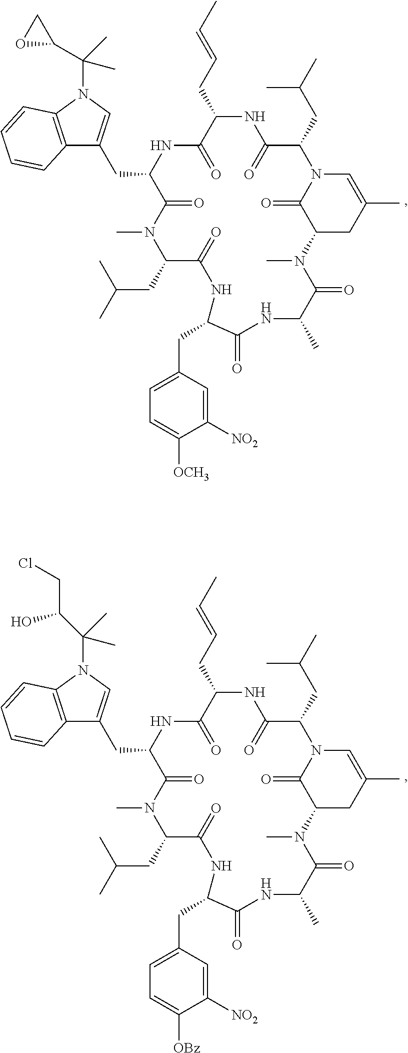

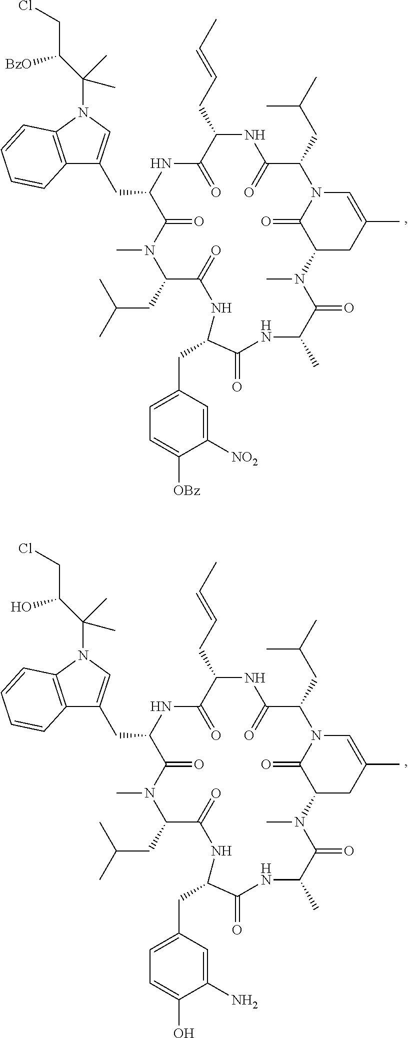

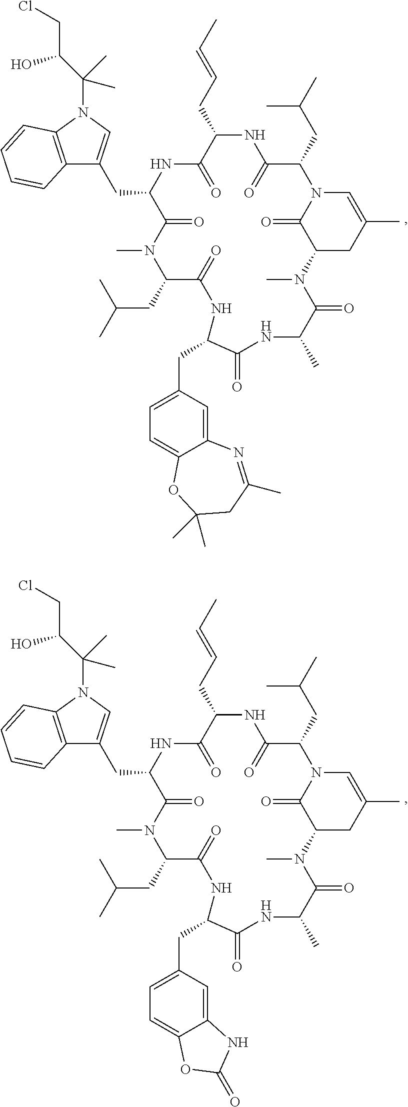 [SMILES: C/C=C/C[C@@H]1NC(=O)[C@H](CC(C)C)N2C=C(C)C[C@@H](C2=O)N(C)C(=O)[C@H](C)NC(=O)[C@H](Cc2ccc3c(c2)N=C(C)CC(C)(C)O3)NC(=O)[C@H](CC(C)C)N(C)C(=O)[C@H](Cc2cn(C(C)(C)[C@H](O)CCl)c3ccccc23)NC1=O ; C/C=C/C[C@@H]1NC(=O)[C@H](CC(C)C)N2C=C(C)C[C@@H](C2=O)N(C)C(=O)[C@H](C)NC(=O)[C@H](Cc2ccc3oc(=O)[nH]c3c2)NC(=O)[C@H](CC(C)C)N(C)C(=O)[C@H](Cc2cn(C(C)(C)[C@H](O)CCl)c3ccccc23)NC1=O]

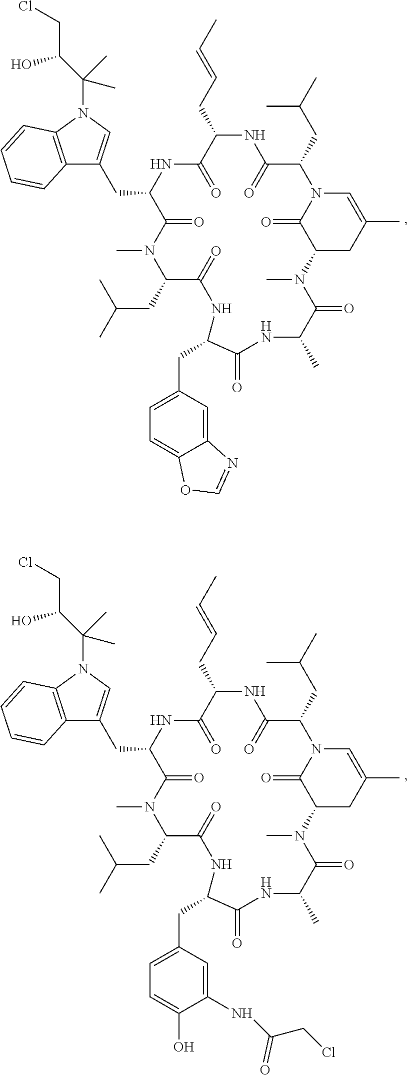 [SMILES: C/C=C/C[C@@H]1NC(=O)[C@H](CC(C)C)N2C=C(C)C[C@@H](C2=O)N(C)C(=O)[C@H](C)NC(=O)[C@H](Cc2ccc3ocnc3c2)NC(=O)[C@H](CC(C)C)N(C)C(=O)[C@H](Cc2cn(C(C)(C)[C@H](O)CCl)c3ccccc23)NC1=O ; C/C=C/C[C@@H]1NC(=O)[C@H](CC(C)C)N2C=C(C)C[C@@H](C2=O)N(C)C(=O)[C@H](C)NC(=O)[C@H](Cc2ccc(O)c(NC(=O)CCl)c2)NC(=O)[C@H](CC(C)C)N(C)C(=O)[C@H](Cc2cn(C(C)(C)[C@H](O)CCl)c3ccccc23)NC1=O]

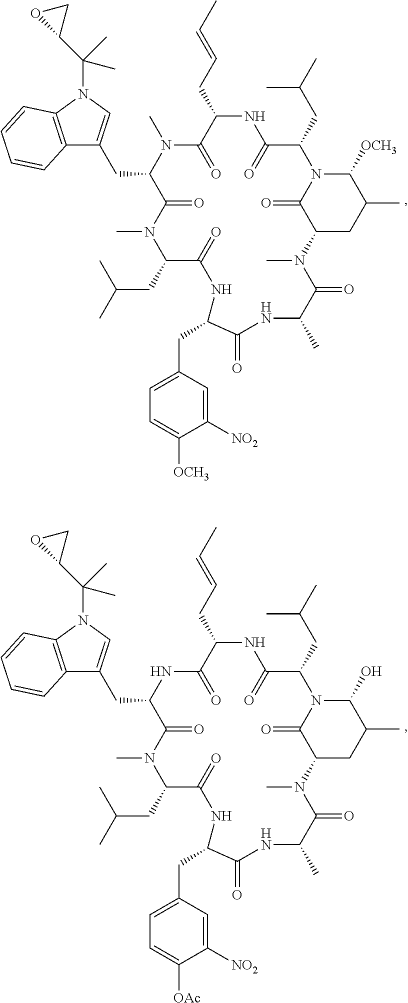 [SMILES: C/C=C/C[C@@H]1NC(=O)[C@H](CC(C)C)N2C(=O)[C@H](CC(C)[C@H]2OC)N(C)C(=O)[C@H](C)NC(=O)[C@H](Cc2ccc(OC)c([N+](=O)[O-])c2)NC(=O)[C@H](CC(C)C)N(C)C(=O)[C@H](Cc2cn(C(C)(C)[C@H]3CO3)c3ccccc23)N(C)C1=O ; C/C=C/C[C@@H]1NC(=O)[C@H](CC(C)C)N2C(=O)[C@H](CC(C)[C@H]2O)N(C)C(=O)[C@H](C)NC(=O)[C@H](Cc2ccc(OC(C)=O)c([N+](=O)[O-])c2)NC(=O)[C@H](CC(C)C)N(C)C(=O)[C@H](Cc2cn(C(C)(C)[C@H]3CO3)c3ccccc23)NC1=O]

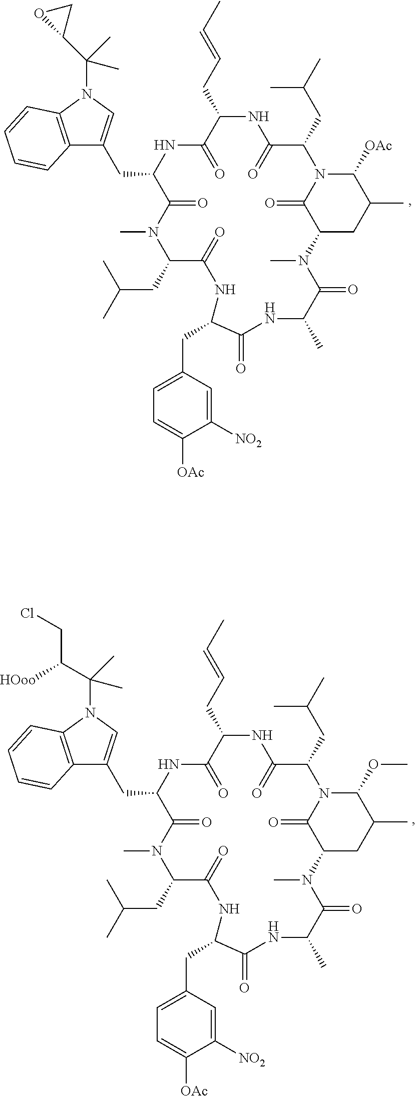

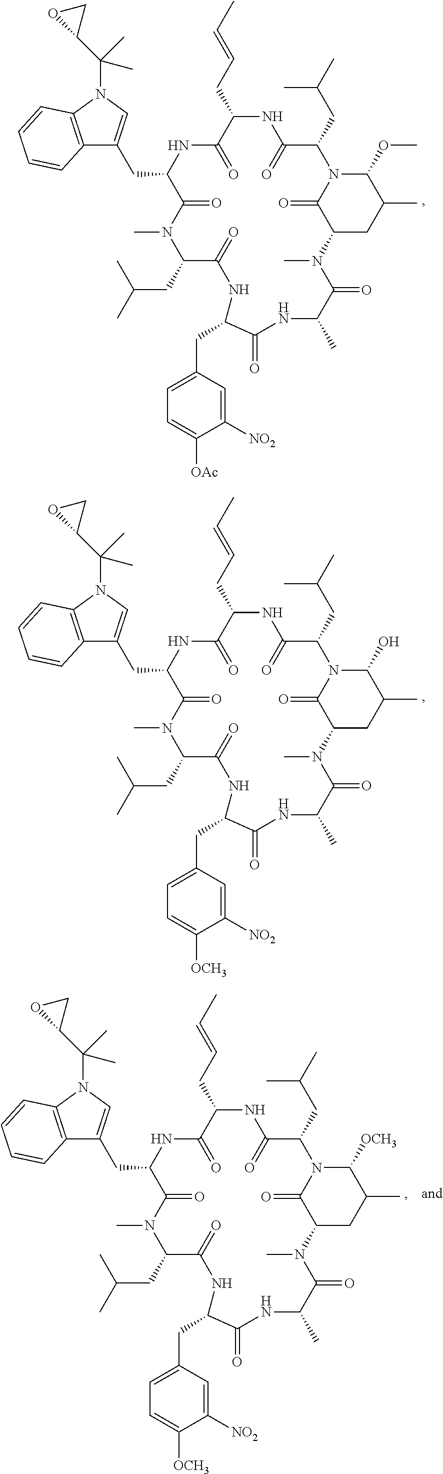

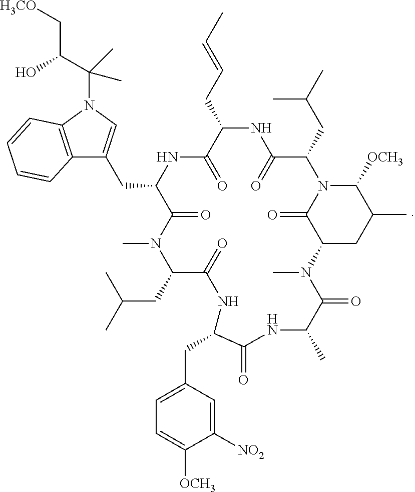 [SMILES: C/C=C/C[C@@H]1NC(=O)[C@H](CC(C)C)N2C(=O)[C@H](CC(C)[C@H]2OC)N(C)C(=O)[C@H](C)NC(=O)[C@H](Cc2ccc(OC)c([N+](=O)[O-])c2)NC(=O)[C@H](CC(C)C)N(C)C(=O)[C@H](Cc2cn(C(C)(C)[C@H](O)COC)c3ccccc23)NC1=O]

The compounds of the disclosure or a pharmaceutically acceptable salt thereof having a structure selected from the group of

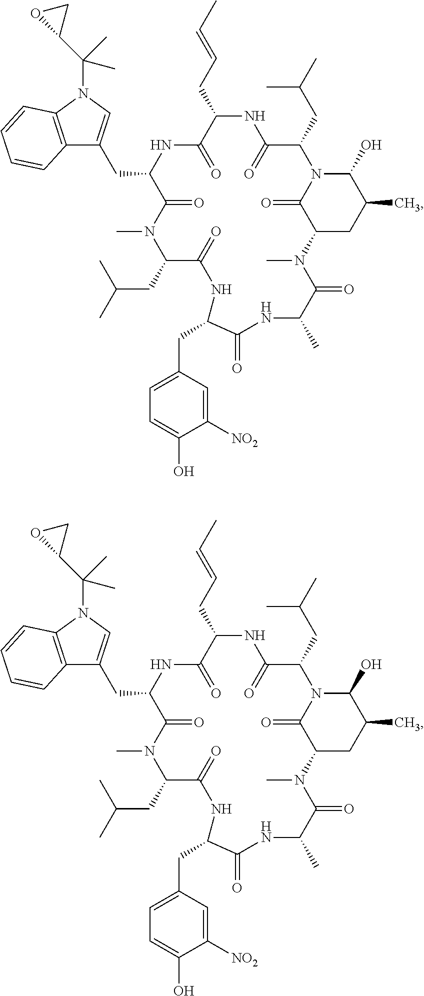 [SMILES: C/C=C/C[C@@H]1NC(=O)[C@H](CC(C)C)N2C(=O)[C@H](C[C@H](C)[C@H]2O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O ; C/C=C/C[C@@H]1NC(=O)[C@H](CC(C)C)N2C(=O)[C@H](C[C@H](C)[C@@H]2O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O]

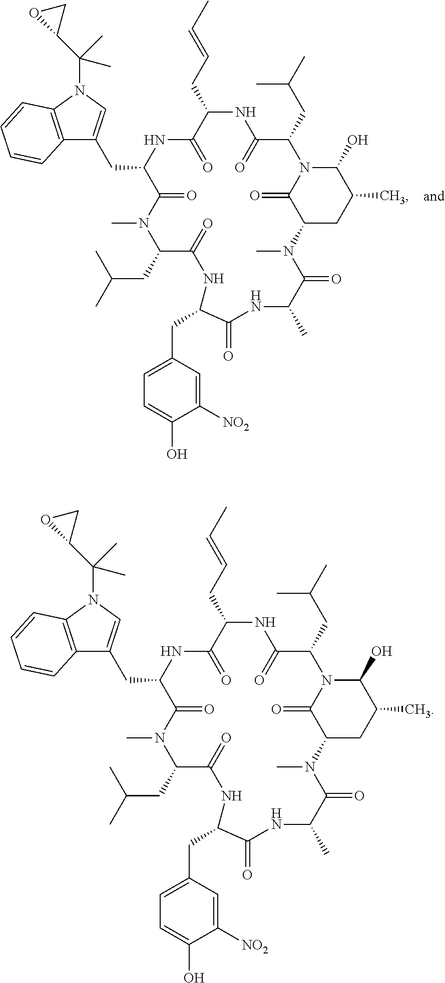 [SMILES: C/C=C/C[C@@H]1NC(=O)[C@H](CC(C)C)N2C(=O)[C@H](C[C@@H](C)[C@H]2O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O ; C/C=C/C[C@@H]1NC(=O)[C@H](CC(C)C)N2C(=O)[C@H](C[C@@H](C)[C@@H]2O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O]

Pharmaceutical Compositions

Provided herein are pharmaceutical compositions comprising a therapeutically effective amount of the compounds of the disclosure, or a pharmaceutically acceptable salt thereof, and an excipient.

The pharmaceutical compositions described herein can comprise any concentration of a compound of the disclosure that is therapeutically effective.

As used herein an “effective” amount or a “therapeutically effective amount” refers to a nontoxic amount of an agent that is effective to provide one or more desired effects in vivo, when administered to a subject in need of treatment. The amount that is “effective” will vary from subject to subject, depending on the age and general condition of the individual, mode of administration, and the like. An “effective” amount in any individual case may be determined using routine experimentation, such as dose-response studies.

In embodiments, the compositions are formulated with one or more pharmaceutically acceptable excipient, carriers, solvents, stabilizers, adjuvants, diluents, or the like, depending upon the particular mode of administration and dosage form. The compositions should generally be formulated to achieve a physiologically compatible pH, and may range from a pH of about 3 to a pH of about 11, preferably about pH 3 to about pH 7, depending on the formulation and route of administration. In alternative embodiments, the pH is adjusted to a range from about pH 5.0 to about pH 8. In embodiments, the compositions may comprise a therapeutically effective amount of the compounds of the disclosure, together with one or more pharmaceutically acceptable excipients. Optionally, the compositions may comprise a combination of the compounds described herein, or may include a second active ingredient useful in the treatment or prevention of bacterial infection (e.g., anti-bacterial or anti-microbial agents).

Compositions disclosed herein, e.g., for parenteral, topical, or oral administration, are most typically solids, liquid solutions, emulsions or suspensions, while inhalable formulations for pulmonary administration are generally liquids or powders. Alternative compositions may be formulated as syrups, creams, ointments, tablets, and the like.

In embodiments, the excipient is a pharmaceutically acceptable excipient. Pharmaceutically acceptable excipients are determined in part by the particular composition being administered, as well as by the particular method used to administer the composition. Accordingly, there exists a wide variety of suitable formulations of pharmaceutical compositions (see, e.g., Remington® Pharmaceutical Sciences).

The term “pharmaceutically acceptable excipient” refers to an excipient for administration of a pharmaceutical agent, such as the compounds described herein. The term refers to any pharmaceutical excipient that may be administered without undue toxicity.

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other excipients include antioxidants (e.g., ascorbic acid), chelating agents (e.g., EDTA), carbohydrates (e.g., dextrin, hydroxyalkylcellulose, and/or hydroxyalkylmethylcellulose), stearic acid, liquids (e.g., oils, water, saline, glycerol and/or ethanol) wetting or emulsifying agents, pH buffering substances, and the like. Liposomes are also included within the definition of pharmaceutically acceptable excipients.

The compositions described herein are formulated in any form suitable for an intended method of administration. When intended for oral use for example, tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use may be prepared according to any method known to the art for the manufacture of pharmaceutical compositions, and such compositions may contain one or more agents including sweetening agents, flavoring agents, coloring agents and preserving agents, in order to provide a palatable preparation.

Pharmaceutically acceptable excipients particularly suitable for use in conjunction with tablets include, for example, inert diluents, such as celluloses, calcium or sodium carbonate, lactose, calcium or sodium phosphate; disintegrating agents, such as cross-linked povidone, maize starch, or alginic acid; binding agents, such as povidone, starch, gelatin or acacia; and lubricating agents, such as magnesium stearate, stearic acid or talc.

Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Compositions for oral use may be also presented as hard gelatin capsules wherein the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil.

The compositions disclosed herein may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such compositions may also contain a demulcent, a preservative, a flavoring or a coloring agent.

Additionally, the compositions may be in the form of a sterile injectable preparation, such as a sterile injectable aqueous emulsion or oleaginous suspension. This emulsion or suspension may be formulated by a person of ordinary skill in the art using those suitable dispersing or wetting agents and suspending agents, including those mentioned above. The sterile injectable preparation may also be a sterile injectable solution or suspension in a nontoxic parenterally acceptable diluent or solvent, such as a solution in 1,2-propane-diol.

Method of Treating

Another aspect of the disclosure provides method of treating a bacterial infection, comprising administering to a subject in need thereof a therapeutically effective amount of a compound of the disclosure, or a pharmaceutically acceptable salt thereof. In a further aspect, the method can be for prophylaxis of a bacterial infection comprising administering a therapeutically effective amount any of the compounds disclosed herein to a subject in need thereof. In embodiments, the method of treating a bacterial infection comprises administering to a subject in need thereof a therapeutically effective amount of a compound of formula I, or a pharmaceutically acceptable salt thereof. In embodiments, the method of treating a bacterial infection comprises administering to a subject in need thereof a therapeutically effective amount of a compound or a pharmaceutically acceptable salt thereof having a structure selected from the group of

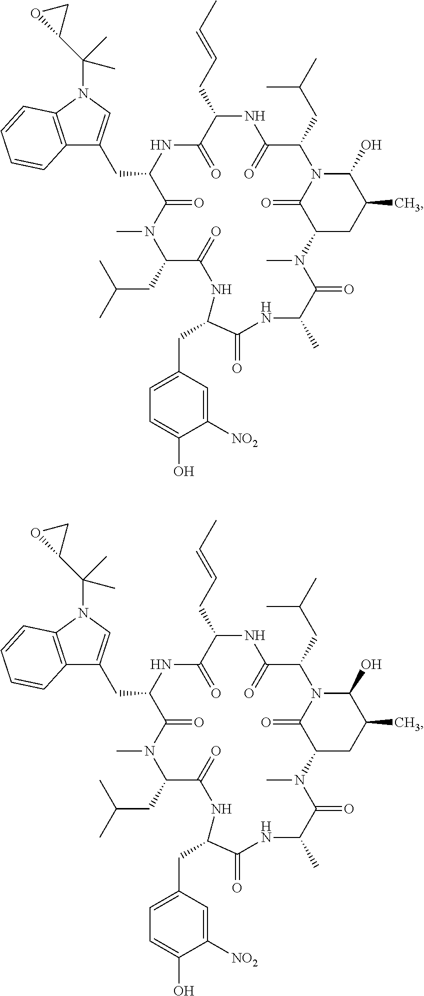 [SMILES: C/C=C/C[C@@H]1NC(=O)[C@H](CC(C)C)N2C(=O)[C@H](C[C@H](C)[C@H]2O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O ; C/C=C/C[C@@H]1NC(=O)[C@H](CC(C)C)N2C(=O)[C@H](C[C@H](C)[C@@H]2O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O]

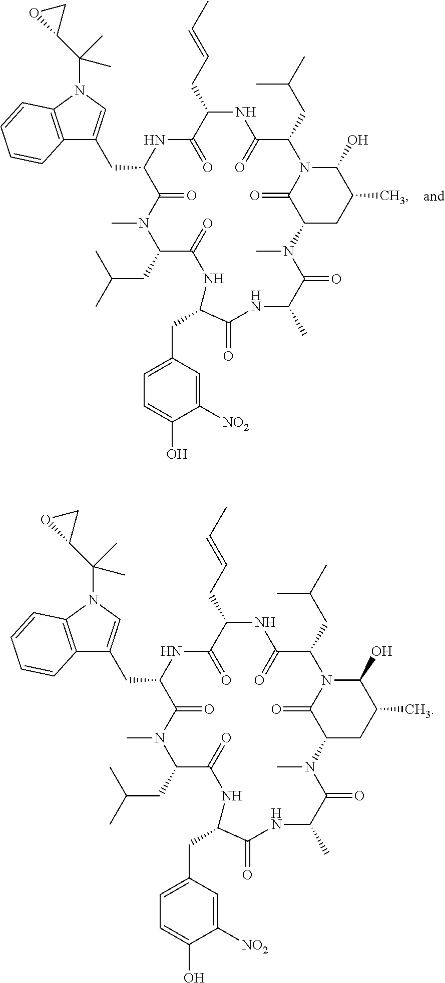 [SMILES: C/C=C/C[C@@H]1NC(=O)[C@H](CC(C)C)N2C(=O)[C@H](C[C@@H](C)[C@H]2O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O ; C/C=C/C[C@@H]1NC(=O)[C@H](CC(C)C)N2C(=O)[C@H](C[C@@H](C)[C@@H]2O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O]

Infections that may be treatable by the compounds of the disclosure can be caused by bacteria. Exemplary microbial infections that may be treated by the methods of the disclosure include, but are not limited to, infections caused by one or more of Staphylococcus aureaus, Enterococcus faecalis, Bacillus anthracis, a Streptococcus species (e.g., Streptococcus pyogenes and Streptococcus pneumoniae), Escherichia coli, Pseudomonas aeruginosa, Burkholderia cepacia, a Proteus species (e.g., Proteus mirabilis and Proteus vulgaris), Klebsiella pneumoniae, Acinetobacter baumannii, Strenotrophomonas maltophillia, Mycobacterium tuberculosis, Mycobacterium bovis, other Mycobacteria of the tuberculosis complex, such as drug resistant strains of tuberculosis, and non-tuberculous Mycobacteria, including Mycobacterium ulcerans.

In embodiments, the bacterial infection is due to Mycobacterium tuberculosis. In embodiments, the Mycobacterium tuberculosis is a drug resistant Mycobacterial strain.

The methods of the disclosure can be useful for treating tuberculosis (TB) in that they inhibit the onset, growth, or spread of a TB infection, cause regression of the TB infection, cure the TB infection, or otherwise improve the general well-being of a subject afflicted with, or at risk of, contracting tuberculosis.

In some embodiments, the compounds of the disclosure have a minimum inhibitory concentration (MIC) against Mycobacterium tuberculosis of 2 μM or less. In embodiments, the compounds of the disclosure have a minimum inhibitory concentration (MIC) against Mycobacterium tuberculosis of 2 μM or less, 1 μM or less, 800 nM or less, 500 nM or less, 300 nM or less, 200 nM or less, 100 nM or less, 80 nM or less, 55 nM or less, 30 nM or less, 20 nM or less, or 10 nM or less. In embodiments, the compounds of the disclosure have a minimum inhibitory concentration (MIC) against Mycobacterium tuberculosis in the range of 1 nM to 1 μM, or 1 nM to 500 nM, or 1 nM to 200 nM, or 1 nM to 100 nM, or 1 nM to 55 nM, or 1 nM to 30 nM, or 1 nM to 10 nM. MICs can be determined via methods known in the art, for example, as described in Hurdle et al., 2008, J. Antimicrob. Chemother. 62:1037-1045.

In some embodiments, the methods of the disclosure further comprise administering to the subject an additional therapeutic compound. In some embodiments, the compound of the invention is administered to the subject before, after, or at the same time as one or more additional therapeutic compounds. In some embodiments, the additional therapeutic compound is an antibiotic. In some embodiments, the additional therapeutic compound is an anti-tuberculosis therapeutic. In some embodiments, the additional therapeutic compound is selected from the group comprising ethambutol, pyrazinamide, isoniazid, levofloxacin, moxifloxacin, gatifloxacin, ofloxacin, kanamycin, amikacin, capreomycin, streptomycin, ethionamide, prothionamide, cycloserine, terididone, para-aminosalicylic acid, clofazimine, clarithromycin, amoxicillin-clavulanate, thiacetazone, meropenem-clavulanate, and thioridazine. In embodiments, the one or more additional therapeutic agent is, for example, an agent useful for the treatment of tuberculosis in a mammal, therapeutic vaccines, antibacterial agents, anti-viral agents; antibiotics and/or agents for the treatment of HIV/AIDS. Examples of such therapeutic agents include isoniazid (INH), ethambutol, rifampin, pirazinamide, streptomycin, capreomycin, ciprofloxacin and clofazimine.

Administration may take the form of single dose administration, or the compositions as disclosed herein can be administered over a period of time, either in divided doses or in a continuous-release formulation or administration method (e.g., a pump). However the compositions of the embodiments are administered to the subject, the amounts of the composition administered and the route of administration chosen should be selected to permit efficacious treatment of the condition.

Compositions disclosed herein, e.g., for parenteral or oral administration, are most typically solids, liquid solutions, emulsions or suspensions, while inhalable formulations for pulmonary administration are generally liquids or powders. Alternative compositions may be formulated as syrups, creams, ointments, tablets, and the like. Compositions disclosed herein can be formulated as a solution that is reconstituted with a physiologically compatible solvent prior to administration for intravenous or intramuscular injection.

The term “pharmaceutically acceptable excipient” refers to an excipient for administration of a pharmaceutical agent, such as the compounds described herein. The term refers to any pharmaceutical excipient that may be administered without undue toxicity.

Pharmaceutically acceptable excipients are determined in part by the particular composition being administered, as well as by the particular method used to administer the composition. Accordingly, there exists a wide variety of suitable formulations of pharmaceutical compositions (see, e.g., Remington® Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other excipients include antioxidants (e.g., ascorbic acid), chelating agents (e.g., EDTA), carbohydrates (e.g., dextrin, hydroxyalkylcellulose, and/or hydroxyalkylmethylcellulose), stearic acid, liquids (e.g., oils, water, saline, glycerol and/or ethanol) wetting or emulsifying agents, pH buffering substances, and the like. Liposomes are also included within the definition of pharmaceutically acceptable excipients.

The compositions described herein are formulated in any form suitable for an intended method of administration. When intended for oral use for example, tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use may be prepared according to any method known to the art for the manufacture of pharmaceutical compositions, and such compositions may contain one or more agents including sweetening agents, flavoring agents, coloring agents and preserving agents, in order to provide a palatable preparation.

Pharmaceutically acceptable excipients particularly suitable for use in conjunction with tablets include, for example, inert diluents, such as celluloses, calcium or sodium carbonate, lactose, calcium or sodium phosphate; disintegrating agents, such as cross-linked povidone, maize starch, or alginic acid; binding agents, such as povidone, starch, gelatin or acacia; and lubricating agents, such as magnesium stearate, stearic acid or talc.

Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Compositions for oral use may be also presented as hard gelatin capsules wherein the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil.

Method of Preparing

Further provided herein are methods for preparing rufomycin analogs. In embodiments, the method comprises admixing a compound having a structure according to Formula (II), an acid, and optionally an alcohol or a C₁-C₃haloalkyl under conditions sufficient to form a compound of formula I; or admixing a compound having a structure according to Formula (II), and one or more reactants selected from the group of a base, an alcohol, a methylating agent, acylating agent, and acyl chloride, under conditions sufficient to form a compound of formula I:

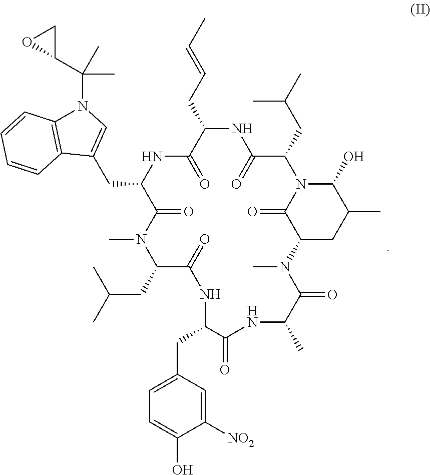 [SMILES: C/C=C/C[C@@H]1NC(=O)[C@H](CC(C)C)N2C(=O)[C@H](CC(C)[C@H]2O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O]

In embodiments, the acid is a strong acid. In embodiments, the acid is HCl or HBr. In embodiments, the acid is NH₄Cl.

In embodiments, the method comprises an alcohol. In embodiments, the alcohol comprises one or more selected from the group of methanol, ethanol, propanol, butanol, pentanol, and hexanol.

In embodiments, the method comprises a C₁-C₃haloalkyl. In embodiments, the C₁-C₃haloalkyl comprises dichloromethane, chloroform, dichloroethylene, methyl iodide, or the like. In embodiments, the C₁-C₃haloalkyl comprises dichloromethane.

In embodiments, the method comprises a base and a methylating agent. In embodiments, the base is a hydroxide salt, such as sodium hydroxide. In embodiments, the methylating agent is dimethyl sulfate. In embodiments, the method further comprises admixing the product of the base and methylating agent with an alcohol, under condition sufficient to form a compound of formula I. In embodiments, the alcohol comprises one or more selected from the group of methanol, ethanol, propanol, butanol, pentanol, and hexanol. In embodiments, the method comprises methanol.

In embodiments, the method comprises a base and an acylating agent. In embodiments, the base is an amine base, such as pyridine or derivatives thereof, triethylamine, piperidine, or the like. In embodiments, the amine base is pyridine. In embodiments, the acylating agent is acetic anhydride. In embodiments, the method further comprises mixing the product of the base and the acylating agent with an acid and an alcohol under condition sufficient to form a compound of formula I. In embodiments, the acid is a strong acid, such as HCl. In embodiments, the alcohol is methanol.

Also provided herein are methods for preparing rufomycin analogs comprising admixing a compound having a structure according to Formula (III), and one or more reactants selected from the group of an acylating agent, benzylating agent, an acid, and a methylating agent under conditions sufficient to form a compound of formula I:

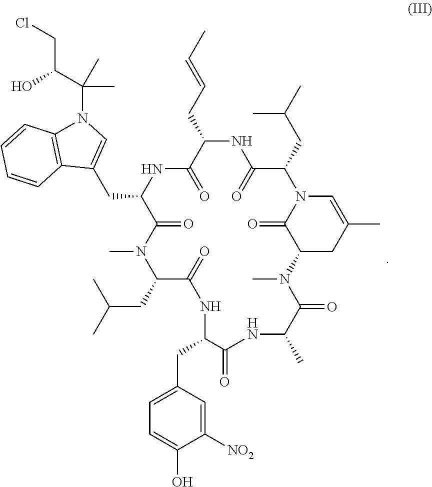 [SMILES: C/C=C/C[C@@H]1NC(=O)[C@H](CC(C)C)N2C=C(C)C[C@@H](C2=O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H](O)CCl)c3ccccc23)NC1=O]

In embodiments the method comprises an acylating agent and a base. In embodiments, the base is an amine base, such as pyridine or derivatives thereof, triethylamine, piperidine, or the like. In embodiments, the amine base is pyridine. In embodiments, the acylating agent is acetic anhydride.

In embodiments, the method comprises a methylating agent. In embodiments, the methylating agent comprises dimethyl sulfate.

In embodiments, the method comprises a benzylating agent. In embodiments, the benzylating agent is benzoyl chloride. In embodiments, the method further comprises a base and a catalyst. In embodiments, the base is an amine base, such as pyridine or derivatives thereof, triethylamine, piperidine, or the like. In embodiments, the amine base is triethylamine. In embodiments, the catalyst is 4-dimethylaminopyridine.

In embodiments, the method comprises an acid. In embodiments, the acid is NH₄Cl. In embodiments, the method further comprises iron. In embodiments, the method further comprises admixing the product of the acid and iron with acetone under condition sufficient to form a compound of formula I. In embodiments, the method further comprises admixing the product of the acid and iron with a base and an acylating agent under conditions sufficient to form a compound of formula I. In embodiment, the base is a hydroxide salt, such as sodium hydroxide. In embodiments, the acylating agent is chloroacetyl chloride. In embodiments, the method further comprises THF, water, or both. In embodiments, the method further comprises admixing the product of the acid and iron with a cyclizing agent under conditions sufficient to form a compound of formula I. In embodiments, the cyclizing agent is 1,1′-carbonyldiimidazole.

Examples of solvents that may be used in the methods of preparing rufomycin analogues of the disclosure include, but are not limited to, organic (e.g., nonpolar aprotic solvents, polar aprotic solvents, or polar protic solvents) and water. The solvents can include one or more of aromatic hydrocarbons, halogenated hydrocarbons, ethers, aliphatic hydrocarbons, or mixtures thereof. The solvents can include alcohols such as methanol, ethanol, propanol, butanol, pentanol, and hexanol, or the like. In embodiments, the solvent is tetrahydrofuran (THF), acetone, water, THF and water, 2-propanol, dichloromethane, methanol, butanol, isopropanol, pentanol, isopropanol, or ethanol. In embodiments, the solvent is a nonpolar aprotic solvent or polar aprotic solvent. In embodiments, the nonpolar aprotic solvent comprises benzene, toluene, hexanes, pentanes, dichloromethane, acetone, trichloromethane, chloro-substituted benzenes, deuterated analogs thereof, or combinations thereof.

The methods of preparing rufomycin analogues of the disclosure can be carried out at, for example, ambient temperatures (e.g., about 20° C. to about 25° C.). Temperatures can be in a range of about 0° C. to about 100° C., about 0° C. to about 35° C., about 5° C. to about 30° C., about 10° C. to about 30° C., about 15° C. to about 25° C., about 20° C. to about 30° C., or about 20° C. to about 25° C., for example, about 0° C., about 5° C., about 10° C., about 15° C., about 20° C., about 25° C., about 30° C., or about 35° C. Reaction times can be instantaneous or otherwise until completion. The progress of the reaction can be monitored by standard techniques, e.g., nuclear magnetic resonance (NMR) spectroscopy. In embodiments, the reaction times are in a range of about 30 seconds to about 72 hours, about 1 minute to about 72 hours, about 5 minutes to about 72 hours, about 10 minutes to about 48 hours, about 15 minutes to about 24 hours, about 1 minute to about 24 hours, about 5 minutes to about 12 hours, about 1 hour to about 6 hours, about 20 minutes to about 1 hour, about 30 minutes (min) to about 12 hours (h), about 1 hour to about 10 hours, about 1 hour to 3 hours, about 25 min to about 6 h, or about 30 min to about 3 h, for example, about 30 seconds, 1 min, 5 min, 10 min, 15 min, 20 min, 25 min, 30 min, 35 min, 40 min, 45 min, 50 min, 55 min, 60 min, 75 min, 90 min, 105 min, 2 h, 3 h, 4 h, 5 h, 6 h, 12 h, 18 h, 24 h, 36 h, 48 h, 60 h, or 72 h.

### EXAMPLES

### Example 1: Synthesis of Rufomycin Analogues 3-8, 10, and 14-31

The schemes below show the synthesis of rufomycin analogues 3-8, 10, and 14-31.

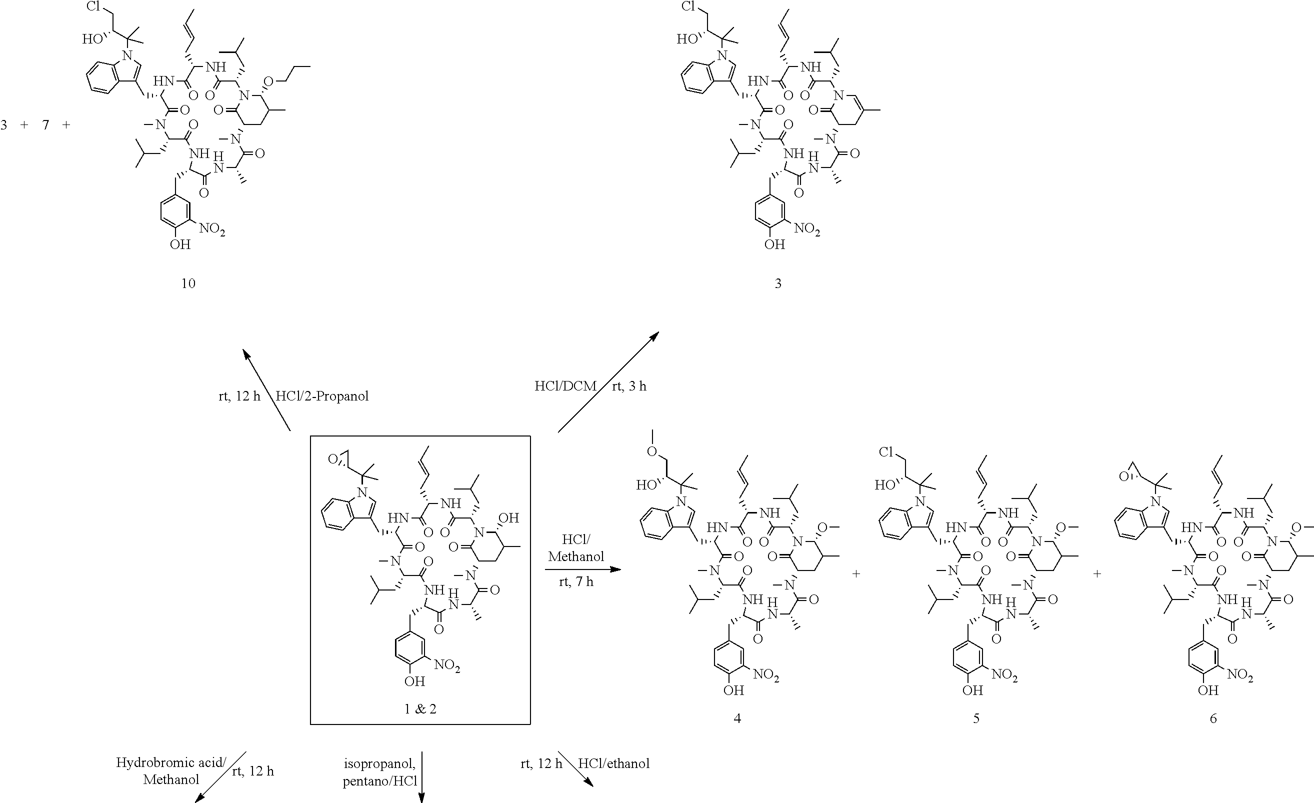

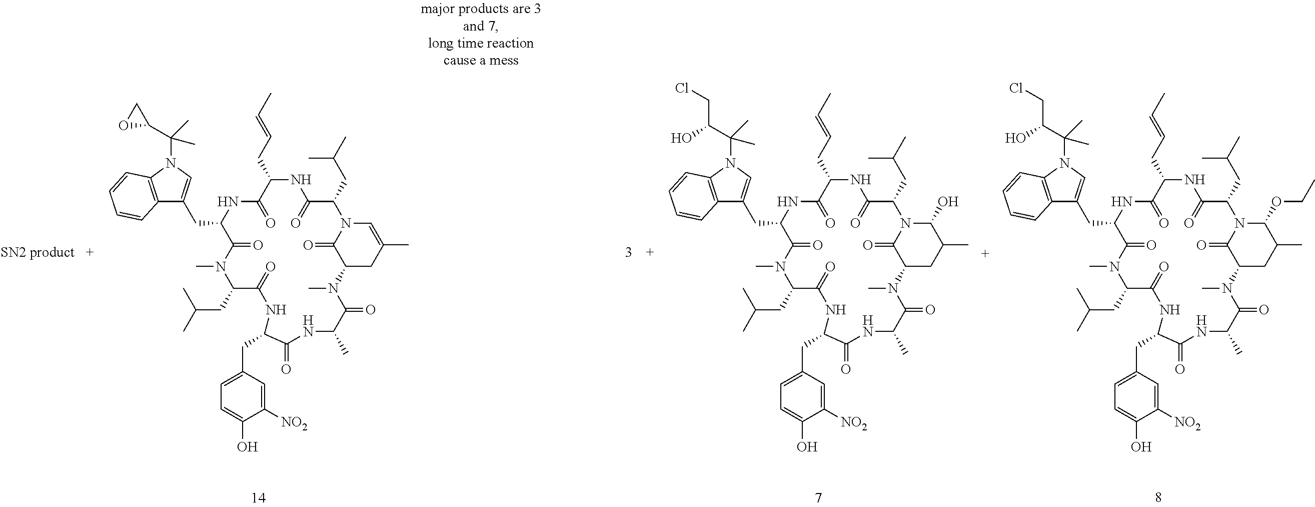

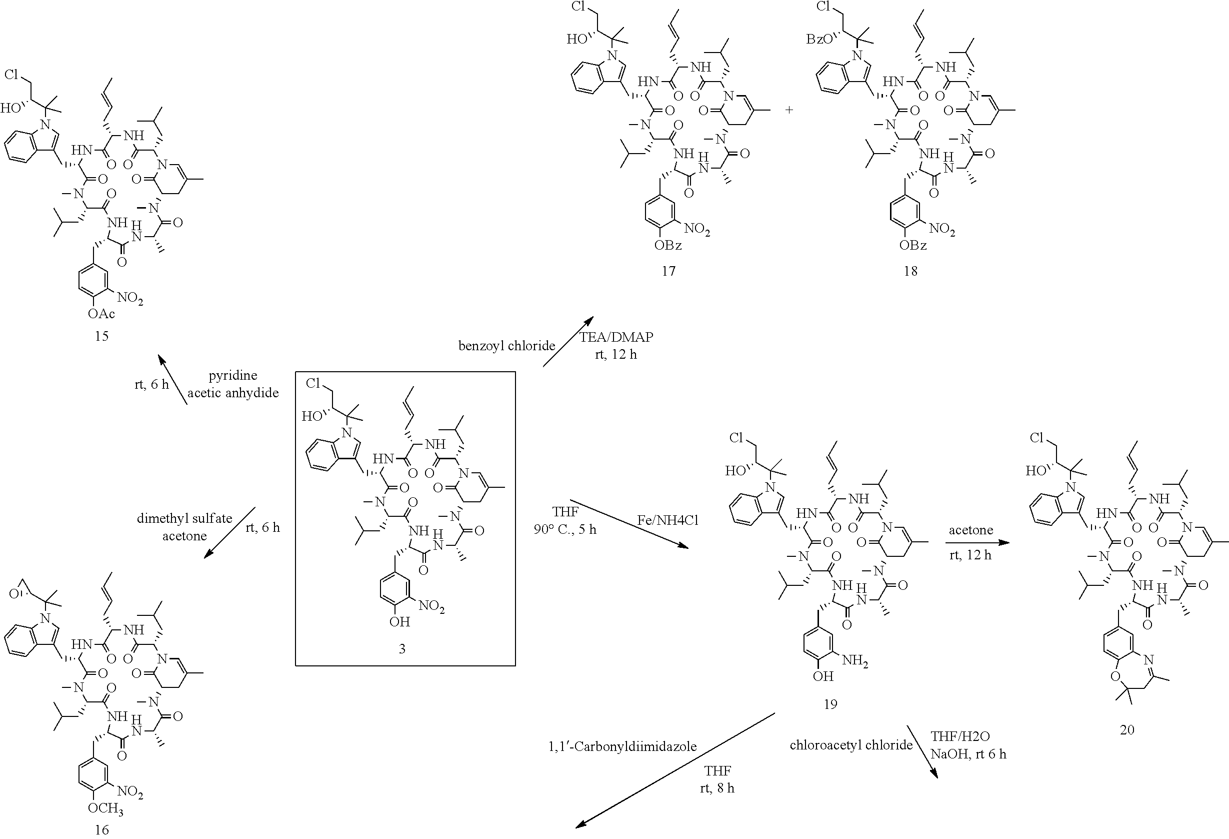

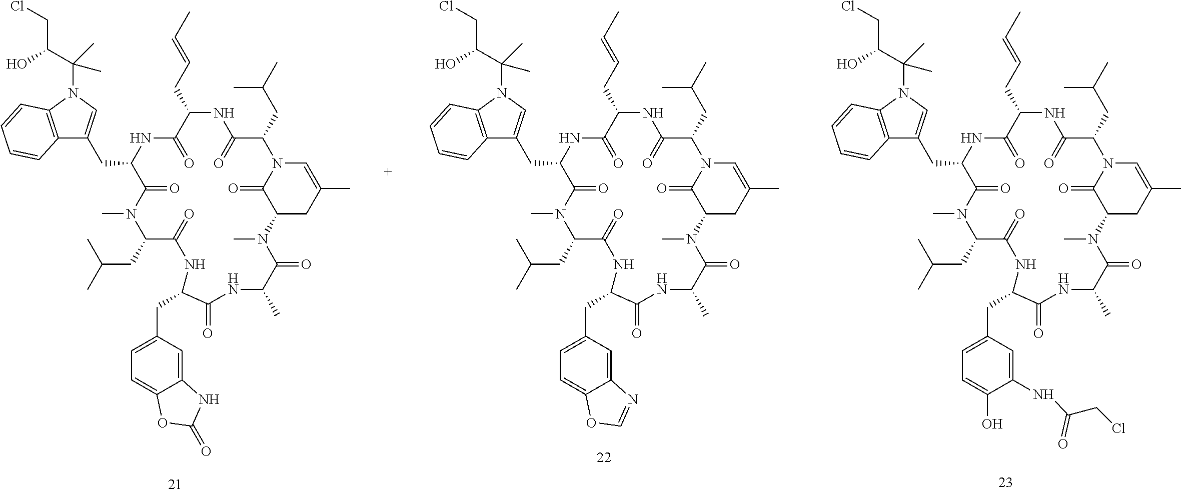

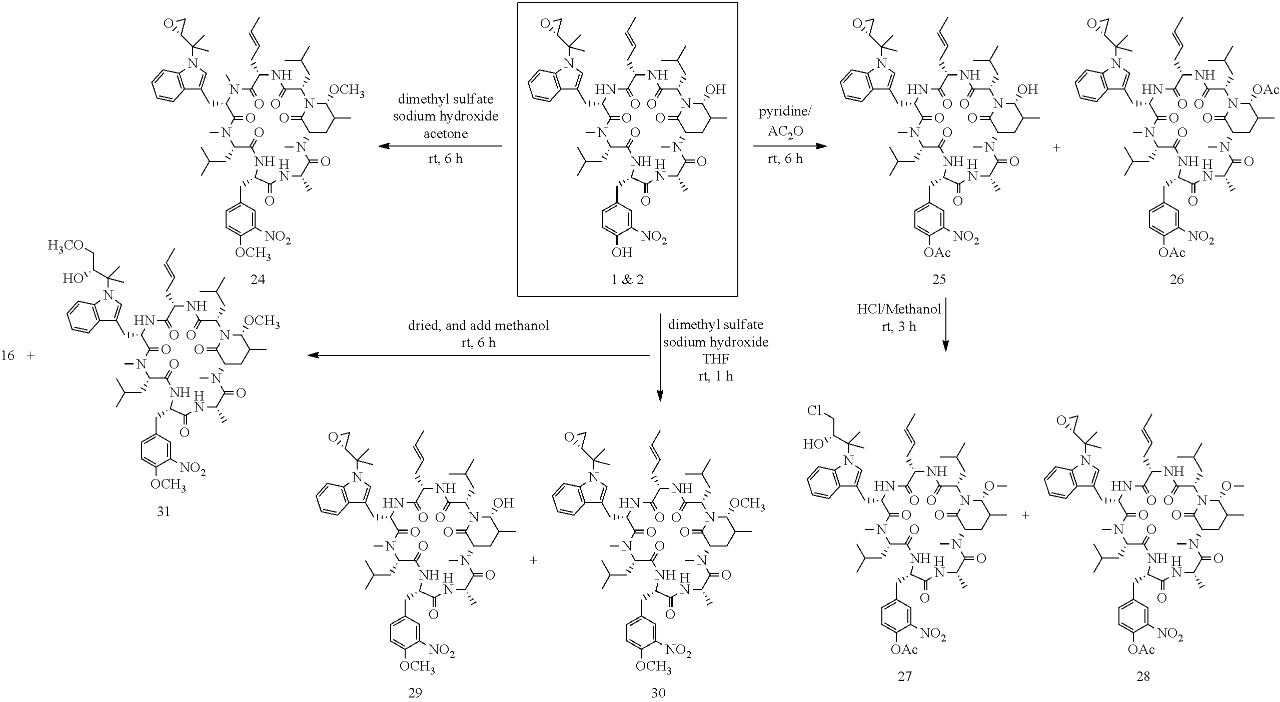

### Example 1: Synthesis of Rufomycin Analogues 3-8, 10, and 14-31

Enrichment of rufomycins 1 and 2 (1 and 2, ˜1:9) mixtures. 28 g of strain 3502 extract was chromatographed on a silica gel CC using a gradient elution of hexane/acetone (7:1, 6:1, 5:1, 4:1, 3:1, 2:1, 1:2, 1:2, and 1:5) to give six fractions (A-F). Fraction C (10 g) with enriched rufomycin 1 and 2 (RUFs I and II), was applied to silica gel CC using a gradient elution of CHCl₃/MeOH (200:1, 100:1, 50:1, 30:1, 20:1, and 10:1) to give five subfractions (C1-C5). Fractions C2-C4 were further purified by Sephadex LH-20 (eluted by ethanol) and/or silica gel CC (eluted with gradient CHCl₃/MeOH), affording a total of ˜6 g of rufomycins 1 and 2 mixtures. Analysis of the NMR and HPLC data revealed the rufomycins 1 and 2 mixtures contained mainly rufomycin 2 (≥90%). (+)-HRESIMS [M+H]⁺ m/z 1042.5559 (calcd for C₅₄H₇₆IN₉O₁₂, 1042.5608).

Preparation of 3. A mixture of RUFs I and 11 (50 mg, 0.048 mmol) was dissolved in 2 ml of DCM. To this solution, 50 μL of 12M hydrogen chloride was added and the reaction mixture was stirred for 3 h. The reaction solvent was then removed under reduced pressure, and the residue was purified by semi-preparative HPLC (68% ACN in H₂O, 2.5 min/mL, 14 min) to give 3, as an amorphous solid (41 mg, 80.7%). (+)-HRESIMS [M+H]⁺ m/z 1060.5231 (calcd for C₅₄H₇₅ClN₉O₁₁, 1060.5269).

Preparation of 4. A mixture of RUFs I and 11 (1, 50 mg, 0.048 mmol) was dissolved in 4 ml of methanol. To this solution, 20 μL of 12M hydrogen chloride was added and the reaction mixture was stirred in room temperature for 7 h. The reaction solvent was then removed under reduced pressure, and the residue was purified by semi-preparative HPLC (68% ACN in H₂O, 2.5 min/mL, 10 min) to give 4 as an amorphous solid (9.6 mg, 18.3%). (+)-HRESIMS [M+H]⁺ m/z 1088.6021 (calcd for C₅₆H₈₂N₉O₁₃, 1088.6027).

Preparation of 5. A mixture of RUFs I and 11 (50 mg, 0.048 mmol) was dissolved in 4 ml of methanol. To this solution, 20 μL of 12M hydrogen chloride was added and the reaction mixture was stirred in room temperature for 7 h. The reaction solvent was then removed under reduced pressure, and the residue was purified by semi-preparative HPLC (68% ACN in H₂O, 2.5 min/mL, 13 min) to give 5 as an amorphous solid (11.1 mg, 21.1%). (+)-HRESIMS [M+H]⁺ m/z 1092.5530 (calcd for C₅₅H₇₉ClN₉O₁₂, 1092.5531).

Preparation of 6. A mixture of RUFs I and 11 (50 mg, 0.048 mmol) was dissolved in 4 ml of methanol. To this solution, 20 μL of 12M hydrogen chloride was added and the reaction mixture was stirred in room temperature for 7 h. The reaction solvent was then removed under reduced pressure, and the residue was purified by semi-preparative HPLC (68% ACN in H₂O, 2.5 min/mL, 16 min) to give 6 as an amorphous solid (13.2 mg, 26.1%). (+)-HRESIMS [M+H]⁺ m/z 1056.5757 (calcd for C₅₅H₇₈N₉O₁₂, 1056.5764).

Preparation of 7. A mixture of RUFs I and II (50 mg, 0.048 mmol) was dissolved in 4 ml of ethanol. To this solution, 20 μL of 12M hydrogen chloride was added and the reaction mixture was stirred in room temperature for 12 h. The reaction solvent was then removed under reduced pressure, and the residue was purified by semi-preparative HPLC (62% ACN in H₂O, 2.5 min/mL, 12 min) to give 7 as an amorphous solid (9.0 mg, 17.4%). (+)-HRESIMS [M+H]⁺ m/z 1078.5351 (calcd for C₅₄H₇₇ClN₉O₁₂, 1078.5375).

Preparation of 8. A mixture of RUFs I and 11 (50 mg, 0.048 mmol) was dissolved in 4 ml of ethanol. To this solution, 20 μL of 12M hydrogen chloride was added and the reaction mixture was stirred in room temperature for 12 h. The reaction solvent was then removed under reduced pressure, and the residue was purified by semi-preparative HPLC (62% ACN in H₂O, 2.5 min/mL, 27 min) to give 8 as an amorphous solid (25.1 mg, 47.1%). (+)-HRESIMS [M+H]⁺ m/z 1106.5715 (calcd for C₅₆H₈₁ClN₉O₁₂, 1106.5688).

Preparation of 10. A mixture of RUFs I and 11 (50 mg, 0.048 mmol) was dissolved in 4 ml of 2-propanol. To this solution, 20 μL of 12M hydrogen chloride was added and the reaction mixture was stirred in room temperature for 12 h. The reaction solvent was then removed under reduced pressure, and the residue was purified by semi-preparative HPLC (68% ACN in H₂O, 2.5 min/mL, 20 min) to give 10 as an amorphous solid (15.1 mg, 9.3%). (+)-HRESIMS [M+H]⁺ m/z 1120.5879 (calcd for C₅₇H₈₃ClN₉O₁₂, 1120.5844).

Preparation of 14. A mixture of RUFs I and 11 (50 mg, 0.048 mmol) was dissolved in 4 ml of methanol. To this solution, 50 μL of 9M hydrogen bromide was added and the reaction mixture was stirred in room temperature for 12 h. The reaction solvent was then removed under reduced pressure, and the residue was purified by semi-preparative HPLC (65% ACN in H₂O, 2.5 min/mL, 23 min) to give 14 as an amorphous solid (3.0 mg, 6.1%). (+)-HRESIMS [M+H]⁺ m/z 1024.5522 (calcd for C₅₄H₇₄N₉O₁₁, 1024.5502).

Preparation of 15. A solution of 3 (50 mg, 0.047 mmol) in 800 μL of pyridine was stirred with acetic anhydride (0.35 mmol, 20 μL) at room temperature for 6 h. The reaction mixture was washed with aqueous HCl and extracted with EtOAc. The combined EtOAc layer was then concentrated under reduced pressure, and the residue was purified by semi-preparative HPLC (65% ACN in H₂O, 2.5 min/mL, 20 min) to give 15 as an amorphous pale-yellow powder (45 mg, 86.6%). (+)-HRESIMS [M+H]⁺ m/z 1102.5389 (calcd for C₅₆H₇₇ClN₉O₁₂, 1102.5375).

Preparation of 16. A solution of 3 (50 mg, 0.047 mmol) in 2 mL of acetone was stirred with sodium hydroxide (0.25 mmol, 10 mg) and dimethyl sulfate (0.84 mmol, 80 μL) at room temperature for 6 h. The reaction was then concentrated under reduced pressure, and the residue was purified by semi-preparative HPLC (68% ACN in H₂O, 2.5 min/mL, 17.5 min) to give 16 as an amorphous powder (15 mg, 30.6%). (+)-HRESIMS [M+H]⁺ m/z 1038.5695 (calcd for C₅₅H₇₆N₉O₁₁, 1038.5659).

Preparation of 17. A solution of 3 (50 mg, 0.047 mmol) in 2 mL of DCM was stirred with dimethylaminopyridine (0.033 mmol, 4 mg) and benzoyl chloride (0.52 mmol, 60 μL), followed by adding 400 μL of triethylamine at room temperature for 6 h. The reaction was then washed by saturated ammonium chloride solution, and the combined DCM layer was concentrated under reduced pressure. Purification of the residue by semi-preparative HPLC (85% ACN in H₂O, 2.5 min/mL, 8 min) afforded 17 as an amorphous powder (5 mg, 9.1%). (+)-HRESIMS [M+H]⁺ m/z 1164.5527 (calcd for C₆₁H₇₉ClN₉O₁₂, 1164.5531).

Preparation of 18. A solution of 3 (50 mg, 0.047 mmol) in 2 mL of DCM was stirred with dimethylaminopyridine (0.033 mmol, 4 mg) and benzoyl chloride (0.52 mmol, 60 μL), followed by adding 400 μL of triethylamine at room temperature for 6 h. The reaction was then washed by saturated ammonium chloride solution, and the combined DCM layer was concentrated under reduced pressure. Purification of the residue by semi-preparative HPLC (85% ACN in H₂O, 2.5 min/mL, 17 min) afforded 18 as an amorphous powder (20 mg, 33.4%). (+)-HRESIMS [M+H]⁺ m/z 1134.6036 (calcd for C₅₈H₈₅ClN₉O₁₂, 1134.6001).

Preparation of 19. To a solution of 3 (50 mg, 0.047 mmol) and iron powder (0.29 mmol, 16 mg) in 5 mL of ethanol, a solution of NH₄Cl (20 mg) in water (400 μL) was added. The reaction mixture was stirred at 90° C. for 4 h. The reaction mixture was then filtered and dried under reduced pressure. The resulting solid was then washed by DCM, and the combined DCM layer was concentrated under reduced pressure. Purification of the residue by semi-preparative HPLC (62% ACN in H₂O, 2.5 min/mL, 10 min) afforded 19 as an amorphous powder (25 mg, 64.2%). (+)-HRESIMS [M+H]⁺ m/z 1030.5531 (calcd for C₅₄H₇₇ClN₉O₉, 1030.5527).

Preparation of 20. 10 mg of 19 were dissolved in 1 mL acetone at room temperature overnight. After removing acetone, methanol was added for semi-prep HPLC analysis and isolation, resulting in the purification (65% ACN in H₂O, 2.5 min/mL, 18 min) of 20 as an amorphous powder (2 mg, 18.5%). (+)-HRESIMS [M+Na]⁺ m/z 1110.6151 (calcd for C₆₀H₈₅ClN₉O₉, 1110.6153).

Preparation of 21. A solution of 19 (30 mg, 0.029 mmol) in 1 mL of THF was stirred with 1,1′-carbonyldiimidazole (0.031 mmol, 5 mg) at room temperature for 8 h. The reaction was then washed by saturated ammonium chloride solution, and the combined DCM layer was concentrated under reduced pressure. Purification of the residue by semi-preparative HPLC (62% ACN in H₂O, 2.5 min/mL, 14 min) afforded 21 as an amorphous powder (4.1 mg, 13.3%). (+)-HRESIMS [M+Na]⁺ m/z 1078.5146 (calcd for C₅₅H₇₄ClN₉O₁₀Na, 1078.5139).

Preparation of 22. A solution of 19 (30 mg, 0.029 mmol) in 1 mL of THF was stirred with 1,1′-carbonyldiimidazole (0.031 mmol, 5 mg) at room temperature for 8 h. The reaction was then washed by saturated ammonium chloride solution, and the combined DCM layer was concentrated under reduced pressure. Purification of the residue by semi-preparative HPLC (62% ACN in H₂O, 2.5 min/mL, 16 min) afforded 22 as an amorphous powder (4.2 mg, 13.9%). (+)-HRESIMS [M+H]⁺ m/z 1040.5396 (calcd for C₅₅H₇₅ClN₉O₉, 1040.5371).

Preparation of 23. To a solution of 19 (30 mg, 0.029 mmol) and chloroacetyl chloride (0.25 mmol, 20 μL) in 2 mL of THF, a solution of Na₂CO₃ (5 mg) in water (100 μL) was added. After stirred at rt for 6 h, the reaction mixture was then concentrated under reduced pressure. Purification of the residue by semi-preparative HPLC (65% ACN in H₂O, 2.5 min/mL, 12 min) afforded 23 as an amorphous powder (4.7 mg, 14.5%). (+)-HRESIMS [M+H]⁺ m/z 1106.5255 (calcd for C₅₆H₇₈ClN₉O₁₀, 1106.5243).

Preparation of 24. A solution of RUFs I and II (50 mg, 0.048 mmol) in 2 mL of acetone was stirred with sodium hydroxide (0.25 mmol, 10 mg) and dimethyl sulfate (0.84 mmol, 80 μL) at room temperature for 6 h. The reaction was then concentrated under reduced pressure, and the residue was purified by semi-preparative HPLC (65% ACN in H₂O, 2.5 min/mL, 32 min) to give 24 as an amorphous powder (5.1 mg, 9.8%). (+)-HRESIMS [M+H]⁺ m/z 1084.6091 (calcd for C₅₇H₃₂N₉O₁₂, 1084.6077).

Preparation of 25. A solution of RUFs I and II (50 mg, 0.048 mmol) in 800 μL of pyridine was stirred with acetic anhydride (0.35 mmol, 20 μL) at room temperature for 6 h. The reaction mixture was washed with aqueous HCl and extracted with EtOAc. The combined EtOAc layer was then concentrated under reduced pressure, and the residue was purified by semi-preparative HPLC (68% ACN in H₂O, 2.5 min/mL, 16 min) to give 25 as an amorphous powder (41 mg, 78.8%). (+)-HRESIMS [M+H]⁺ m/z 1084.5745 (calcd for C₅₆H₇₈N₉O₁₃, 1084.5714).

Preparation of 26. A solution of 1 (50 mg, 0.048 mmol) in 800 μL of pyridine was stirred with acetic anhydride (0.35 mmol, 20 μL) at room temperature for 6 h. The reaction mixture was washed with aqueous HCl and extracted with EtOAc. The combined EtOAc layer was then concentrated under reduced pressure, and the residue was purified by semi-preparative HPLC (68% ACN in H₂O, 2.5 min/mL, 28 min) to give 26 as an amorphous pale-yellow powder (4.5 mg, 8.3%). (+)-HRESIMS [M+H]⁺ m/z 1126.5848 (calcd for C₅₈H₃₀N₉O₁₄, 1126.5819).

Preparation of 27. To a solution of 25 (10 mg, 0.009 mmol) in 1 mL methanol, 10 μL of 12M hydrogen chloride was added and the reaction mixture was stirred in room temperature for 3 h. The reaction solvent was then removed under reduced pressure, and the residue was purified by semi-preparative HPLC (65% ACN in H₂O, 2.5 min/mL, 19 min) to give 27 as an amorphous powder (3.2 mg, 31%). (+)-HRESIMS [M+H]⁺ m/z 1134.5632 (calcd for C₅₇H₃₁ClN₉O₁₃, 1134.5637).

Preparation of 28. To a solution of 25 (10 mg, 0.009 mmol) in 1 mL methanol, 10 μL of 12M hydrogen chloride was added and the reaction mixture was stirred in room temperature for 3 h. The reaction solvent was then removed under reduced pressure, and the residue was purified by semi-preparative HPLC (65% ACN in H₂O, 2.5 min/mL, 24 min) to give 28 as an amorphous powder (2.9 mg, 29%). (+)-HRESIMS [M+Na]⁺ m/z 1120.5684 (calcd for C₅₇H₇₉N₉O₁₃Na, 1120.5690).

Preparation of 29. A solution of RUFs I and II (50 mg, 0.048 mmol) in 2 mL of acetone was stirred with sodium hydroxide (0.25 mmol, 10 mg) and dimethyl sulfate (0.84 mmol, 80 μL) at room temperature for 6 h. The reaction was then concentrated under reduced pressure, and the residue was purified by semi-preparative HPLC (65% ACN in H₂O, 2.5 min/mL, 13 min) to give 29 as an amorphous powder (17.7 mg, 35%). (+)-HRESIMS [M+H]⁺ m/z 1056.5767 (calcd for C₅₅H₇₈N₉O₁₂, 1056.5764).

Preparation of 30. A solution of RUFs I and II (50 mg, 0.048 mmol) in 2 mL of acetone was stirred with sodium hydroxide (0.25 mmol, 10 mg) and dimethyl sulfate (0.84 mmol, 80 μL) at room temperature for 6 h. The reaction was then concentrated under reduced pressure, and the residue was purified by semi-preparative HPLC (65% ACN in H₂O, 2.5 min/mL, 22 min) to give 30 as an amorphous powder (23.1 mg, 40%). (+)-HRESIMS [M+H]⁺ m/z 1070.5960 (calcd for C₅₆H₇₉N₉O₁₂, 1070.5921).

Preparation of 31. A solution of RUFs I and II (50 mg, 0.048 mmol) in 4 mL of THF was stirred with sodium hydroxide (0.25 mmol, 10 mg) and dimethyl sulfate (0.84 mmol, 80 μL) at room temperature for 1 h. The reaction was then concentrated under reduced pressure and stirred with 4 mL of methanol for 5 h. The reaction was then concentrated under reduced pressure, and the residue was purified by semi-preparative HPLC (65% ACN in H₂O, 2.5 min/mL, 12 min) to give 31 as an amorphous powder (21.2 mg, 40%). (+)-HRESIMS [M+H]⁺ m/z 1134.6036 (calcd for C₅₈H₈₅ClN₉O₁₂, 1134.6001).

MICs against M. tuberculosis. The MIC was defined as the minimum concentration of the compound required to achieve a reduction in fluorescence by 90% relative to the untreated bacterial controls. The anti-TB activity was determined by the microplate Alamar Blue assay (MABA) as previously described (Choules et al., Antimicrob. Agents Chemother. 2019, 63, e02204-02218).

Analysis of binding affinity to Mycobacterial ClpC1-NTD and FL by SPR. SPR binding assay was performed using a previously reported method (Choules et al., Antimicrob. Agents Chemother. 2019, 63, e02204-02218; Wolf et al., ACS Infect. Dis. 2019, 5, 829-840). Kinetic rate constants (kₐ and k_{d}) were determined by fitting the double-reference data globally to the 1:1 Langmuir model embedded in the Biacore T200 evaluation software (v3.0). K_{D} values were then calculated from the two rate constants (K_{D}=k_{d}/kₐ). Smaller K_{D} values represent tighter binding affinities.

Anti-Mycobacterium tuberculosis (Mtb) Bioactivity Profiles and SAR Considerations. Anti-M.tb activities and binding affinities (K_{D}) with both caseinolytic protein C1 (ClpC1) N-terminal domain (NTD) and the full-length protein (FL) by surface plasmon resonance (SPR) of rufomycins 1-3, 4-8, 10, and 14-31, see Table 1.

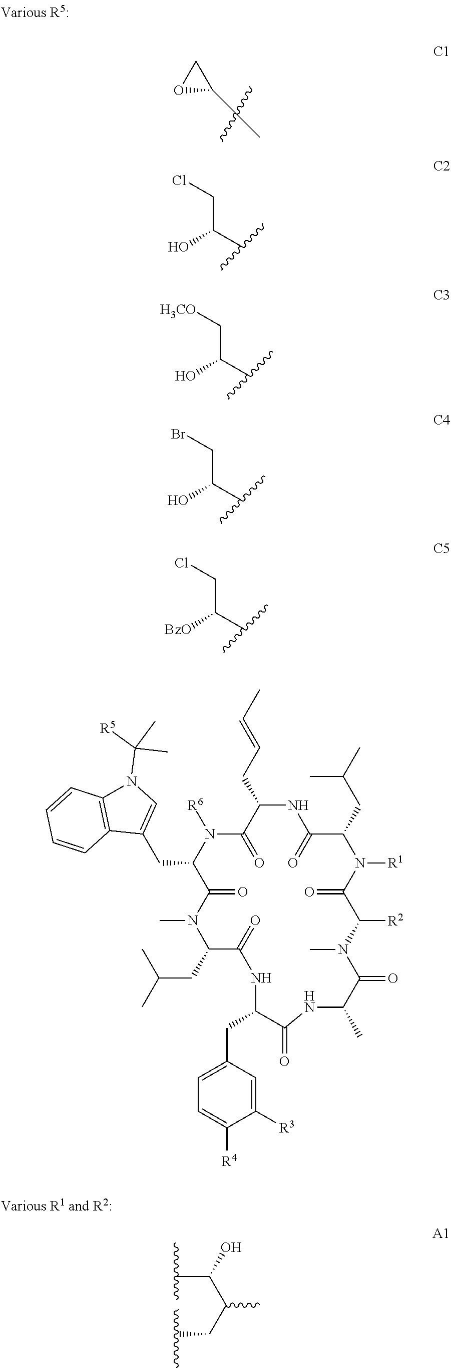

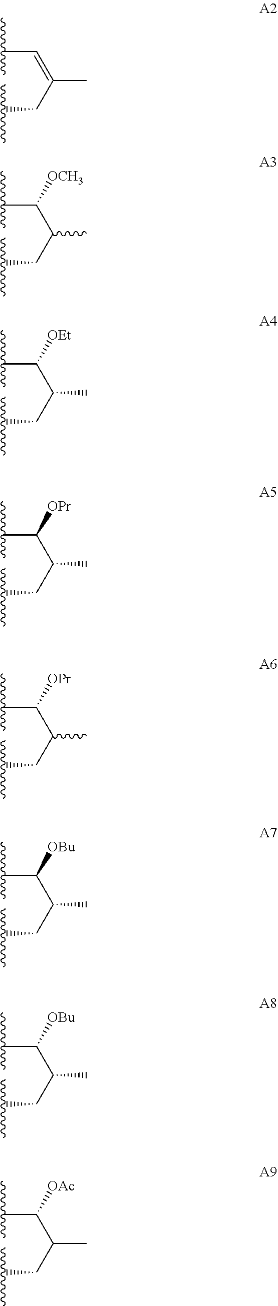

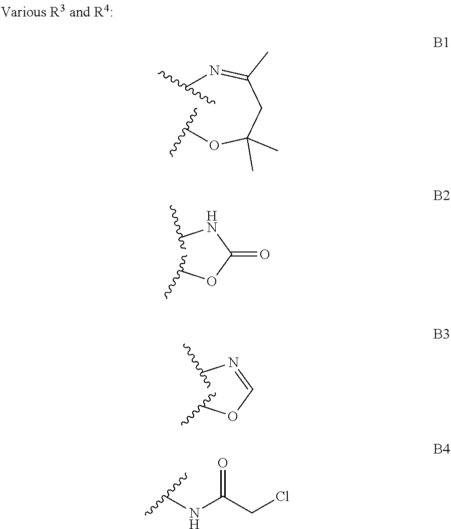

TABLE 1

| | | | | | | | | | |
|---|---|---|---|---|---|---|---|---|---|
| | | | | | | | MIC | SPR KD NTD | SPR KD FL |
| Compound | R¹ | R² | R³ | R⁴ | R⁵ | R⁶ | (nM) | (nM) | (nM) |

| 1 + 2 | A1 | NO₂ | OH | C1 | H | 10 ± 1 | 70 | 70 |
|---|---|---|---|---|---|---|---|---|
| 3 | A2 | NO₂ | OH | C2 | H | 18.8 ± 4 | 82.3 | 97.1 |
| 4 | A3 | NO₂ | OH | C3 | H | 6.4 ± 1.8 | 88.5 | 79.9 |
| 5 | A3 | NO₂ | OH | C2 | H | 7.3 ± (<1) | 123.3 | 163.3 |
| 6 | A3 | NO₂ | OH | C1 | H | 1.9 ± (<1) | 102.6 | 31.7 |
| 7 | A1 | NO₂ | OH | C2 | H | 46.4 ± 4.6 | 114.5 | 115.8 |
| 8 | A4 | NO₂ | OH | C2 | H | 244.0 ± 27.1 | 120.4 | 141.7 |
| 10 | A6 | NO₂ | OH | C2 | H | 749.5 ± 10.7 | 90.7 | 142.7 |
| 14 | A2 | NO₂ | OH | C1 | H | | | |
| 15 | A2 | NO₂ | OAc | C2 | H | 22.4 | 317.1 | 512.6 |
| 16 | A2 | NO₂ | OCH₃ | C1 | H | 72.6 | 579.6 | 883.2 |
| 17 | A2 | NO₂ | OBz | C2 | H | 52.9 | 1246.5 | 2027.0 |
| 18 | A2 | NO₂ | OBz | C5 | H | 399.9 | 1425.5 | 1142.0 |
| 19 | A2 | NH₂ | OH | C2 | H | 887.1 | 500.9 | 319.3 |
| 20 | A2 | B1 | | C2 | H | 782 | 195.6 | 233.6 |
| 21 | A2 | B2 | | C2 | H | | | |
| 22 | A2 | B3 | | C2 | H | | | |
| 23 | A2 | B4 | OH | C2 | H | | | |
| 24 | A3 | NO₂ | OCH₃ | C1 | CH₃ | 64.6 | 450.6 | 513.2 |
| 25 | A1 | NO₂ | OAc | C1 | H | 18.4 ± 6 | 56.0 | 72.5 |
| 26 | A9 | NO₂ | OAc | C1 | H | 8.9 ± 3.6 | 33.8 | 16.0 |
| 27 | A3 | NO₂ | OAc | C1 | H | 1.4 | 77.6 | 121.2 |
| 28 | A3 | NO₂ | OAc | C2 | H | 5.1 | 62.1 | 97.7 |
| 29 | A1 | NO₂ | OCH₃ | C1 | H | 54.3 | 116.7 | 132.9 |
| 30 | A3 | NO₂ | OCH₃ | C1 | H | 5.8 | 55.5 | 75.1 |
| 31 | A3 | NO₂ | OCH₃ | C3 | H | 23.3 | 57.4 | 67.9 |

Table 1 shows that there are 10 rufomycin analogues that have an MIC of less than 28 nM and also have good binding affinity. There are also 4 rufomycin analogues of the disclosure that have an MIC of less than 10. Rufomycin analogues 6 and 27 have MICs of 1.9 and 1.4 respectively, a ten-fold increase in activity compared to rufomycin analogues 1 and 2.

### Example 2—Isolation and Elucidation of Rufomycin Diastereomers 32-35

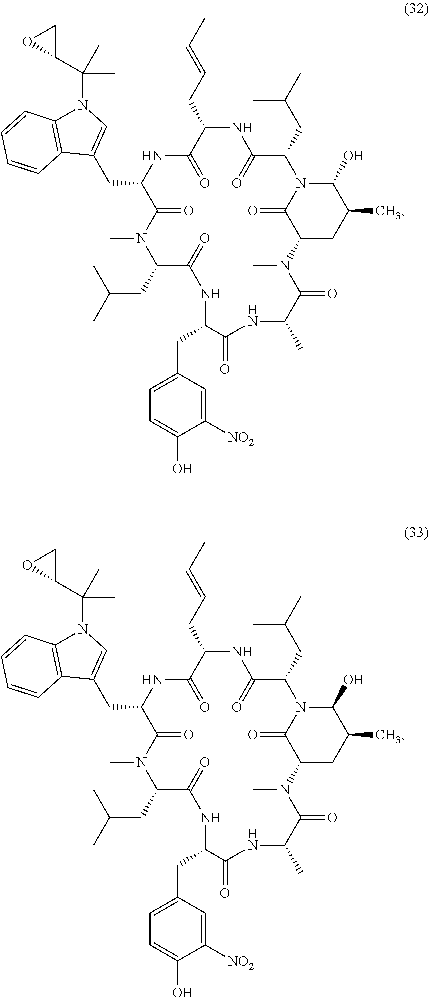 [SMILES: C/C=C/C[C@@H]1NC(=O)[C@H](CC(C)C)N2C(=O)[C@H](C[C@H](C)[C@H]2O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O ; C/C=C/C[C@@H]1NC(=O)[C@H](CC(C)C)N2C(=O)[C@H](C[C@H](C)[C@@H]2O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O]

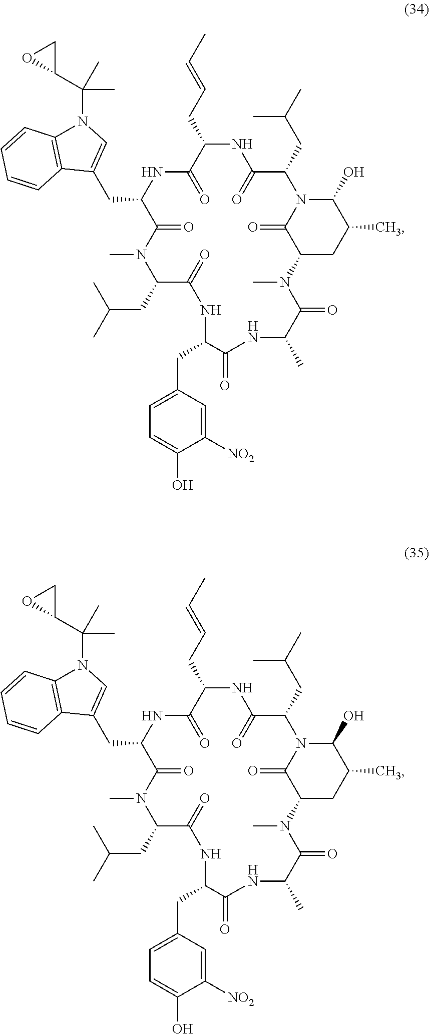 [SMILES: C/C=C/C[C@@H]1NC(=O)[C@H](CC(C)C)N2C(=O)[C@H](C[C@@H](C)[C@H]2O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O ; C/C=C/C[C@@H]1NC(=O)[C@H](CC(C)C)N2C(=O)[C@H](C[C@@H](C)[C@@H]2O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O]

General Experimental Procedures. ESI-MS/MS spectra were carried out by using Bruker Impact II, quadrupole time of flight (q-TOF) equipped with a Shimadzu UHPLC (Kyoto, Japan). The ion source was operated in the positive electrospray ionization mode using capillary voltage at 4.0 kV; nebulizer and drying gas (N₂) at 0.4 bar and 4.0 L/min, respectively; dry temperature of 225° C.; and mass scan range set from m/z 50 to 2000. The separation was performed on a CORTECS C18 (100×3.0 mm, 2.7 m) UPLC column. Data were collected and processed by the Data Analysis 4.4 software (Bruker Daltonik GmbH, Germany). All 1D/2D NMR spectra were acquired on a JEOL (Jeol Resonance Inc., Peabody, Mass., USA) ECZ 400 MHz or Bruker Ultrashield 600 Plus with AVANCE III console 600 MHz spectrometer (Bruker, Billerica, Mass.). The acquired spectra were processed using the Mnova NMR software package (v.12.0.4, MestReLab Research S. L., A Coruna, Spain). Sephadex LH-20 (Pharmacia, Uppsala, Sweden) and Silica gel (ICN EcoChrom 32-63, 60 Å) was used for column chromatography. Semi-preparative HPLC was performed on a Shimadzu HPLC (Kyoto, Japan) connected to a PDA detector (Shimadzu, model SPD-20A) and equipped with a Kinetex EVO C18 (250×10 mm, S-5, 100 Å) column. TLC was analyzed by UV detector and vanillin-sulfuric acid spray (3 g vanillin, 95 ml ethanol, and 1.5 ml sulfuric acid). All solvents used were obtained from Fisher Scientific (Fair Lawn, N.J., USA) or Sigma-Aldrich (St. Louis, Mo., USA).

Strain Material. The strain MJM3502 was obtained from the Extract Collection of Useful Microorganisms (ECUM) at Myongji University, Republic of Korea. The Streptomyces strain MJM3502 was shown to be 99% identical to Streptomyces atratus (NRRL B-16927; identical to the ATCC strain) through classification using 16S rDNA sequence and phylogenetic analysis (Choules et al., J. Org. Chem. 2018, 83, 6664-6672; Zhou et al., J. Nat. Prod. 2020, 83, 657-667). Strain MJM3502 showed similar morphology to S. atratus NRRL: B-16927 with gray to pale yellow aerial mycelium on ISP2-SP4 medium, and the growth was robust. However, in ISP5 medium, the growth was poor compared to S. atratus NRRL-B-16927.

Extraction and Isolation. MJM3502 whole broth (300 L) was treated the same way as previously reported (Zhou et al., J. Nat. Prod. 2020, 83, 657-667), affording the 3502 ethyl acetate (3502 EA) fraction. A rufomycin enriched fraction (28 g) was obtained from 3502 EA extract by Silica gel CC with n-hexane/EA (5:5) and ethyl acetate as eluent. The rufomycin enriched fraction was further chromatographed on silica gel using a gradient elution of n-hexane/acetone (7:1, 6:1, 5:1, 4:1, 3:1, 2:1, 1:1, 1:2, and 1:5) to give six fractions (A-F). About 6 g of rufomycins 32 and 34 mixtures were enriched from fraction C (10 g) by a series of columns packed with silica gel (CHCl₃/MeOH) and Sephadex LH-20 (MeOH or EtOH), and rufomycins 32-35 and a secondary rufomycin analogue were obtained by semi-preparative HPLC (60% ACN in H₂O, 2.5 mL/min) from the remaining material of fraction C.

Rufomycin 32: pale yellow, amorphous solid; ¹H and ¹³C NMR (CD₃OD) see Table 1. (+)-HRESIMS m/z 1042.5632 [M+H]⁺ (calcd for C₄H₇₆N₉O₁₂, 1042.5608).

Rufomycin 33: pale yellow, amorphous solid; ¹H (600 MHz, CD₃OD) and ¹³C NMR (125 MHz, CD₃OD) see Table 1. (+)-HRESIMS m/z 1042.5608 [M+H]⁻ (calcd for C₅₄H₇₆N₉O₁₂, 1042.5608).

Rufomycin 34: pale yellow, amorphous solid; ¹H (600 MHz, CD₃OD) and ¹³C NMR (125 MHz, CD₃OD) see Table 1. (+)-HRESIMS m/z 1042.5608 [M+H]⁺ (calcd for C₆₄H₇₆N₉O₁₂, 1042.5608).

Rufomycin 35: pale yellow, amorphous solid; ¹H (600 MHz, CD₃OD) and ¹³C NMR (125 MHz, CD₃OD) see Table 1. (+)-HRESIMS m/z 1044.5782 [M+H]+(calcd for C₅₄H₇₇N₉O₁₂, 1044.5764).

The rufomycins 32-35 were identified as four diastereomers, arising from different orientations of the hydroxyl and methyl groups in the hydroxy-methyl piperidinone moiety. Rufomycin 32 was assigned the (AA5, 4S,5R) absolute configuration based on the ROESY correlations, and further confirmed by the cocrystal structures of rufomycin 32 and ClpC1-NTD. Thus, rufomycin 32 is that congener previously identified as ilamycin, Compound 4, and ilamycin C1 (PCT/US2000/015044). HPLC analysis showed rufomycins 32 and 33 are interconvertible, indicating rufomycin 34 to be an epimer of rufomycin 32 at the hemi-aminal carbon, based on the expected property of hemi-aminals. The assigned 4S,5S (AA5) absolute configuration for rufomycin 33 was validated by the ROESY correlations. Rufomycin 34 with the absolute configuration 4R,5R (AA5) was established by the ROESY cross-peaks. Finally, the fourth diastereoisomer rufomycin 35 was isolated and identified. rufomycin 34 was found to convert slowly to a small amount of rufomycin 35 (˜5%) under aqueous conditions. Rufomycin 35, the most unstable diastereomer, quickly epimerized to rufomycin 34 (˜50%) during concentration. After being purified by semi-prep HPLC, rufomycin 35 was concentrated under a stream of forced air and freeze drying to reduce interconversion. The ¹H and ¹³C NMR spectra (Table 2) showed co-existence of both rufomycins 34 and 35 (˜2:5). The ROESY correlations confirmed that rufomycin 35 had a 4R,5S (AA5) absolute configuration.

TABLE 2
¹H (600 MHz) and ¹³C NMR (125 MHz) data of rufomycins 32-35 in CD₃OD.

| | rufomycin 32 | | rufomycin 33 | | rufomycin 34 | | rufomycin 35 | |
|---|---|---|---|---|---|---|---|---|
| | δ_{H}, mult. | | δ_{H}, mult. | | δ_{H}, mult. | | δ_{H}, mult. | |
| No. | (J in Hz) | δ_{C} | (J in Hz) | δ_{C} | (J in Hz) | δ_{C} | (J in Hz) | δ_{C} |
| AA1-1 | | 174.2 | | 174.3 | | 174.2 | | 174.0 |
| 2 | 4.87, dd | 51.7 | 4.90, m | 51.9 | 4.87, dd | 51.6 | 4.96, m | 51.5 |
| | (9.3, 6.5) | | | | (9.7, 5.9) | | | |
| 3 | 3.22, m | 29.2 | 3.22, m | 29.1 | 3.22, m | 29.2 | 3.23, m | 29.2 |
| 4 | 7.18, s | 125.9 | 7.15, s | 125.8 | 7.17, s | 125.8 | 7.17, s | 125.7 |
| 5 | | 109.1 | | 109.0 | | 109.0 | | 109.0 |
| 6 | | 130.6 | | 130.5 | | 130.6 | | 130.6 |
| 7 | 7.52, br d | 119.8 | 7.51, m | 119.8 | 7.51, br d | 119.8 | 7.53, m | 119.5 |
| | (7.9) | | | | (7.9) | | | |
| 8 | 7.06, m | 120.5 | 7.06, m | 120.5 | 7.04, m | 120.5 | 7.04, m | 120.5 |
| 9 | 7.12, m | 122.6 | 7.13, m | 122.6 | 7.11, m | 122.6 | 7.13, m | 122.6 |
| 10 | 7.76, br d | 114.6 | 7.76, m | 114.6 | 7.75, br d | 114.5 | 7.77, m | 114.7 |
| | (8.5) | | | | (8.5) | | | |
| 11 | | 137.1 | | 137.0 | | 137.0 | | 137.1 |
| 1′ | | 59.2 | | 59.2 | | 59.1 | | 59.2 |
| 2′ | 3.24, dd | 58.9 | 3.23, dd | 58.9 | 3.23, dd | 58.9 | 3.24, dd | 58.9 |
| | (4.1, 2.7) | | (4.1, 2.6) | | (4.1, 2.6) | | (4.1, 2.6) | |
| 3′a | 2.81, dd | 46.0 | 2.80, dd | 46.0 | 2.81, dd | 46.0 | 2.81, dd | 46.0 |
| | (−4.6, 2.7) | | (−4.6, 2.6) | | (−4.6, 2.7) | | (−4.6, 2.6) | |
| 3′b | 2.85, dd | | 2.85, dd | | 2.85, dd | | 2.86, dd | |
| | (−4.6, 4.1) | | (−4.6, 4.1) | | (−4.6, 4.1) | | (−4.6, 4.1) | |
| 1″ | 1.51, s | 23.2 | 1.49, s | 23.2 | 1.49, s | 23.2 | 1.51, s | 23.2 |
| 1″′ | 1.66, s | 25.0 | 1.65, s | 25.0 | 1.64, s | 25.0 | 1.66, s | 25.0 |
| AA2-1 | | 169.9 | | 169.8 | | 169.8 | | 170.0 |
| 2 | 4.27, dd | 59.6 | 4.31, dd | 59.5 | 4.27, dd | 59.6 | 4.46, dd | 60.0 |
| | (10.7, 3.8) | | (11.2, 3.2) | | (10.8, 3.6) | | (11.0, 3.2) | |
| 3a | 1.51, m | 37.7 | 1.50, m | 37.6 | 1.50, m | 37.7 | 1.47 | 37.7 |
| 3b | −0.51, m | | −0.65, m | | −0.49, m | | −0.65, m | |
| 4 | 0.94, m | 25.6 | 0.94, m | 25.5 | 0.93, m | 25.5 | 0.99, m | 25.5 |
| 5 | 0.10, d | 21.5 | 0.07, d | 21.3 | 0.09, d | 21.4 | 0.15, d | 21.2 |
| | (6.6) | | (6.6) | | (6.6) | | (6.6) | |
| 6 | 0.42, d | 23.3 | 0.38, d | 23.4 | 0.42, d | 23.3 | 0.38, d | 23.3 |
| | (6.6) | | (6.6) | | (6.6) | | (6.6) | |
| NMe | 2.34, s | 29.4 | 2.30, s | 29.3 | 2.34, s | 29.4 | 2.13, s | 29.2 |
| AA3-1 | | 171.7 | | 171.6 | | 171.6 | | 171.4 |
| 2 | 4.66, m | 57.1 | 4.68, m | 56.8 | 4.66, m | 57.0 | 4.66, m | 54.2 |
| 3a | 3.07, dd | 38.3 | 3.06, dd | 38.5 | 3.08, dd | 38.2 | 3.13, m | 39.1 |
| | (−14.2, 5.7) | | (−14.1, 5.8) | | (−14.1, 5.9) | | | |
| 3b | 2.87, m | | 2.86, m | | 2.87, m | | 2.87, m | |
| 4 | | 130.1 | | 129.9 | | 130.1 | | 131.2 |
| 5 | 7.82, d | 126.4 | 7.77, d | 126.6 | 7.83, d | 126.4 | 7.92, d | 126.7 |
| | (2.2) | | (2.2) | | (2.2) | | (2.2) | |
| 6 | | 135.5 | | 135.4 | | 135.5 | | 135.5 |
| 7 | | 154.4 | | 154.4 | | 154.3 | | 154.1 |
| 8 | 7.06, d | 121.2 | 7.06, d | 121.3 | 7.06, d | 121.2 | 7.03, d | 120.8 |
| | (8.6) | | (8.6) | | (8.6) | | (8.6) | |
| 9 | 7.38, dd | 138.9 | 7.36, dd | 138.8 | 7.38, dd | 138.9 | 7.36, dd | 139.0 |
| | (8.6, 2.2) | | (8.6, 2.2) | | (8.6, 2.2) | | (8.6, 2.2) | |
| AA4-1 | | 172.7 | | 173.5 | | 172.5 | | 174.4 |
| 2 | 4.80, q | 47.9 | 4.80, q | 47.8 | 4.79, q | 47.7 | 5.04, q | 44.7 |
| | (6.6) | | (6.6) | | (6.6) | | (6.6) | |
| 3 | 1.28, d | 17.9 | 1.29, d | 17.8 | 1.27, d | 17.8 | 1.35, d | 17.9 |
| | (6.6) | | (6.6) | | (6.6) | | (6.6) | |
| AA5-1 | | 171.3 | | 170.4 | | 171.9 | | 171.6 |
| 2 | 3.82, dd | 60.2 | 3.81, d | 60.1 | 3.77, dd | 63.1 | 5.60, m | 60.4 |
| | (9.9, 7.1) | | (9.0) | | (11.2, 7.1) | | | |
| 3α | 2.55. ddd | 26.4 | 1.71, m | 31.8 | 2.27, m | 26.8 | 1.90, m | 32.0 |
| | (−13.4, 10.1, 3.9) | | | | | | | |
| 3β | 1.87, m | | 2.36, m | | 1.86, m | | 1.90, m | |
| 4 | 2.22, m | 35.1 | 2.63, m | 33.7 | 1.96, m | 34.2 | 1.93, m | 37.2 |
| 5 | 4.76, br d | 81.7 | 4.63, br d | 82.5 | 4.76, br d | 79.4 | 4.34, br d | 91.2 |
| | (3.70) | | (2.6) | | (2.6) | | (7.9) | |
| 1′ | 1.10, d | 16.3 | 0.98, d | 17.4 | 1.08, d | 17.5 | 1.20, d | 17.8 |
| | (7.2) | | (6.7) | | (6.8) | | (5.8) | |
| NMe | 3.24, s | 38.3 | 3.23, s | 38.8 | 3.24, s | 38.4 | 2.71, s | 30.6 |
| AA6-1 | | 173.4 | | 172.9 | | 173.1 | | 174.4 |
| 2 | 5.24, dd | 55.0 | 5.46, dd | 56.9 | 5.24, dd | 55.1 | 3.97, dd | 65.2 |
| | (11.9, 4.3) | | (11.0, 4.1) | | (11.1, 5.4) | | (9.19, 6.1) | |
| 3a | 1.89, ddd | 35.8 | 1.89, m | 37.6 | 1.93, m | 35.8 | 1.81, m | 39.9 |
| | (−14.9, 11.9, 3.6) | | | | | | | |
| 3b | 1.96, ddd | | 1.95, m | | 1.96, m | | 2.26, m | |
| | (14.9, 10.8, 4.3) | | | | | | | |
| 4 | 1.51, m | 25.6 | 1.82, m | 25.5 | 1.39, m | 25.8 | 1.61, m | 26.2 |
| 5 | 0.93, d | 21.3 | 0.95, d | 21.8 | 0.90, d | 21.3 | 0.97, d | 22.6 |
| | (6.5) | | (6.5) | | (6.5) | | (6.5) | |
| 6 | 1.01, d | 23.9 | 0.97, d | 23.8 | 1.01, d | 23.9 | 0.99, d | 23.2 |
| | (6.5) | | (6.5) | | (6.5) | | (6.5) | |
| AA7-1 | | 173.4 | | 173.3 | | 173.3 | | 173.3 |
| 2 | 4.61, m | 54.3 | 4.78, m | 53.4 | 4.63, m | 54.3 | 4.70, m | 53.1 |
| 3a | 2.78, m | 35.3 | 2.65, m | 35.6 | 2.79, m | 35.2 | 2.64, m | 39.3 |
| 3b | 2.62, m | | 2.60, m | | 2.62, m | | 2.34, m | |
| 4 | 5.59, m | 127.7 | 5.62, m | 127.5 | 5.57, m | 127.5 | 5.48, m | 126.7 |
| 5 | 5.61, m | 129.4 | 5.62, m | 129.3 | 5.63, m | 129.4 | 5.52, m | 130.3 |
| 6 | 1.65, m | 18.3 | 1.69, m | 18.5 | 1.65, m | 18.4 | 1.61, m | 18.2 |

MICs against M. tuberculosis. The MIC was defined as the minimum concentration of the compound required to achieve a reduction in fluorescence by 90% relative to the untreated bacterial controls. The anti-TB activity was determined by the microplate Alamar Blue assay (MABA) as previously described (Choules et al., Antimicrob. Agents Chemother. 2019, 63, e02204-02218).

Analysis of binding affinity to Mycobacterial ClpC1-NTD and FL by SPR. SPR binding assay was performed using a previously reported method (Choules et al., Antimicrob. Agents Chemother. 2019, 63, e02204-02218; Wolf et al., ACS Infect. Dis. 2019, 5, 829-840). Kinetic rate constants (kₐ and k_{d}) were determined by fitting the double-reference data globally to the 1:1 Langmuir model embedded in the Biacore T200 evaluation software (v3.0). K_{D} values were then calculated from the two rate constants (K_{D}=k_{d}/kₐ). Smaller K_{D} values represent tighter binding affinities.

Anti-Mycobacterium tuberculosis (Mtb) Bioactivity Profiles and SAR Considerations. Anti-M.tb activities and binding affinities (K_{D}) with both caseinolytic protein C1 (ClpC1) N-terminal domain (NTD) and the full-length protein (FL) by surface plasmon resonance (SPR) of rufomycins 32-35 (Table 3). Based on the nature of hemi-aminal, rufomycins 32 and 33, and rufomycins 34 and 35 are supposed to be similar mixtures of corresponding epimers in the test medium. Similar K_{D} and MIC values for rufomycins 32 and 33 and for rufomycins 34 and 35 supported that rufomycins with an S configuration at C4 in AA5 are preferred for anti-M.tb activity. The MIC data shows that rufomycin 32 and 33 have higher potency than drugs rifampin and isoniazid for anti-M.tb activity.

TABLE 3

| | | K_{D} | K_{D} |
|---|---|---|---|
| Compound | MIC₉₀ | NTD | FL |
| rufomycin 32 | 21 | 32 | 31 |
| rufomycin 33 | 12 | 29 | 27 |
| rufomycin 34 | 46 | 183 | 178 |
| rufomycin 35 | 47 | 186 | 209 |
| RMP | 28 | NT | NT |
| INH | 463 | NT | NT |

NT = not tested;
RMP = rifampin and
INH = isoniazid

What is claimed:

1. A compound having a structure according to Formula I or a pharmaceutically acceptable salt thereof: 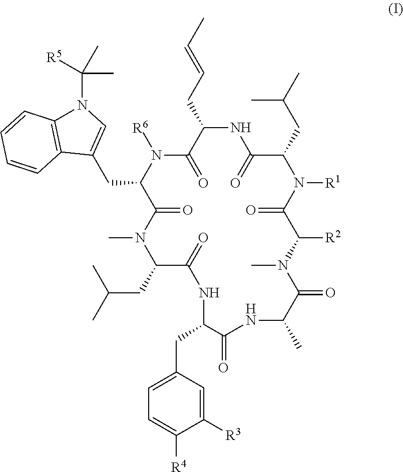
wherein,
R¹ and R², together are selected from 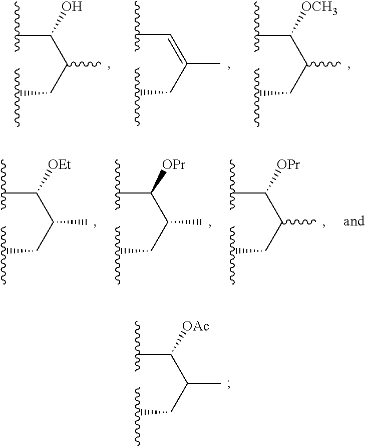
R³ and R⁴ are independently selected from NO₂, OH, OAc, OCH₃, OBz, NH₂, and 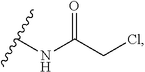 [SMILES: *NC(=O)CCl]
or R³ and R⁴ together with the carbon atoms to which they are attached, form a five- to seven-membered substituted or unsubstituted heterocycloalkyl comprising 1 to 3 ring heteroatoms selected from O and N;
R⁵ is selected from 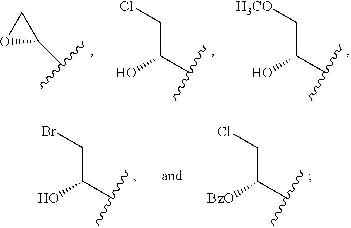
and,
R⁶ is selected from H and C₁-C₃ alkyl;
provided when R¹ and R² together are 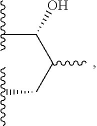
R³ is NO₂, R⁴ is OH, and R⁵ is 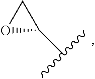 [SMILES: *[C@H]1CO1]
that R⁶ is not H; when R¹ and R² together are 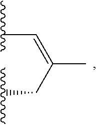
R³ is NO₂, R⁴ is OH, and R⁵ is 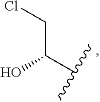 [SMILES: *[C@H](O)CCl]
that R⁶ is not H; when R¹ and R² together are 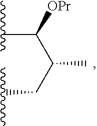
R³ is NO₂, R⁴ is OH, and R⁵ is 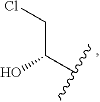 [SMILES: *[C@H](O)CCl]
that R⁶ is not H; and when R¹ and R² together are 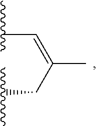
R³ is NO₂, R⁴ is OH, and R⁵ is 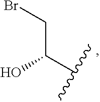 [SMILES: *[C@H](O)CBr]
that R⁶ is not H.

2. The compound of claim 1, wherein R¹ and R² together are selected from

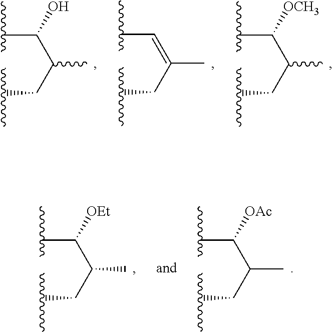

3. The compound of claim 2, wherein R¹ and R² together are selected from

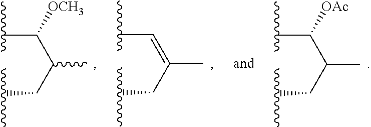

4. The compound of claim 1, wherein R³ is NO₂, NH₂, or

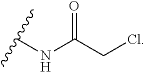 [SMILES: *NC(=O)CCl]

5. The compound of claim 4, wherein R³ is NO₂.

6. The compound of claim 1, wherein R⁴ is OH, OAc, OCH₃, or OBz.

7. The compound of claim 1, wherein R⁴ is OH, OAc, or OCH₃.

8. The compound of claim 1, wherein R³ and R⁴ together are selected from

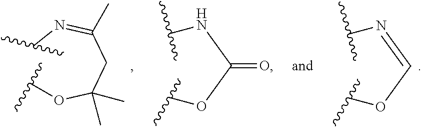

9. The compound of claim 1, wherein R⁵ is selected from

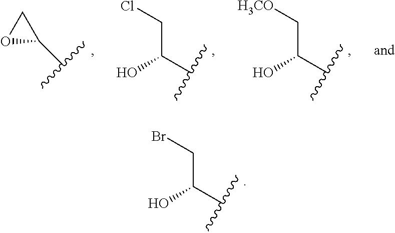

10. The compound of claim 1, wherein R⁶ is H or CH₃.

11. The compound of claim 1, wherein R⁶ is H.

12. The compound of claim 1 selected from the group of:

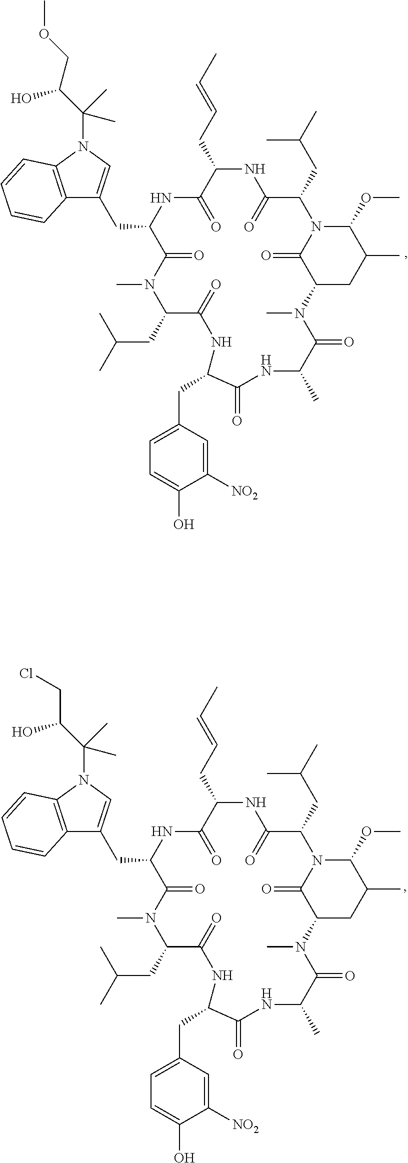 [SMILES: C/C=C/C[C@@H]1NC(=O)[C@H](CC(C)C)N2C(=O)[C@H](CC(C)[C@H]2OC)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H](O)COC)c3ccccc23)NC1=O ; C/C=C/C[C@@H]1NC(=O)[C@H](CC(C)C)N2C(=O)[C@H](CC(C)[C@H]2OC)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H](O)CCl)c3ccccc23)NC1=O]

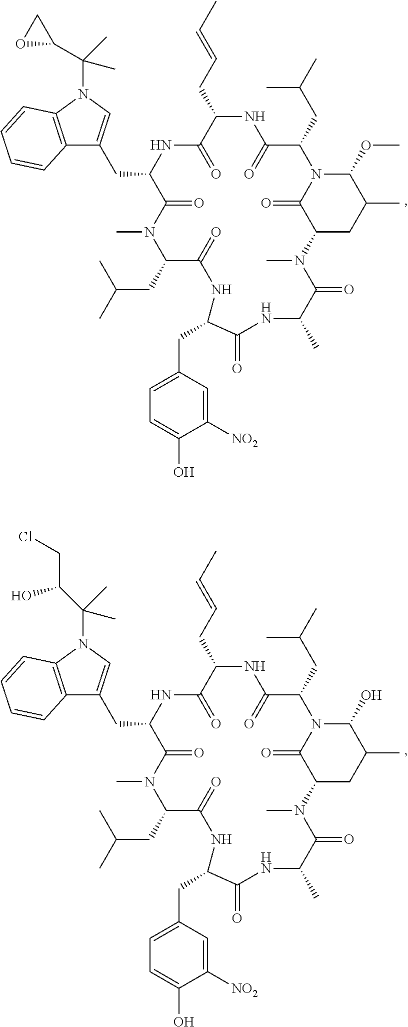 [SMILES: C/C=C/C[C@@H]1NC(=O)[C@H](CC(C)C)N2C(=O)[C@H](CC(C)[C@H]2OC)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O ; C/C=C/C[C@@H]1NC(=O)[C@H](CC(C)C)N2C(=O)[C@H](CC(C)[C@H]2O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H](O)CCl)c3ccccc23)NC1=O]

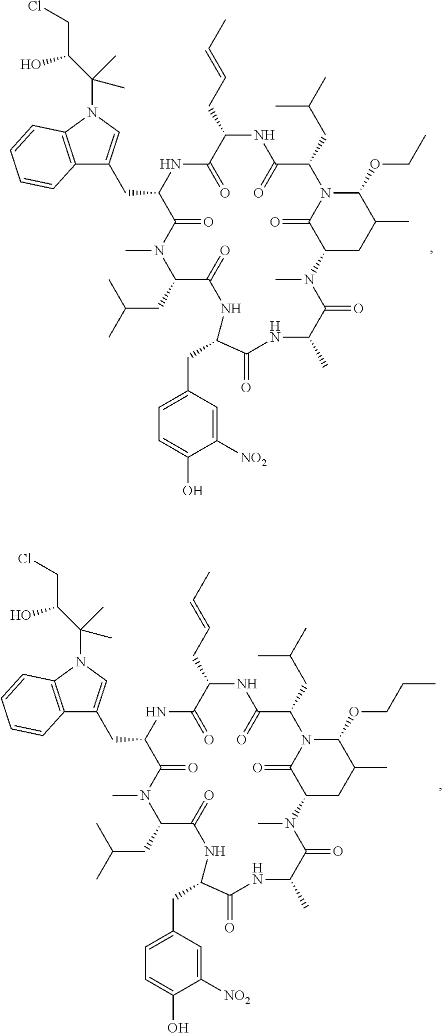 [SMILES: C/C=C/C[C@@H]1NC(=O)[C@H](CC(C)C)N2C(=O)[C@H](CC(C)[C@H]2OCC)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H](O)CCl)c3ccccc23)NC1=O ; C/C=C/C[C@@H]1NC(=O)[C@H](CC(C)C)N2C(=O)[C@H](CC(C)[C@H]2OCCC)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H](O)CCl)c3ccccc23)NC1=O]

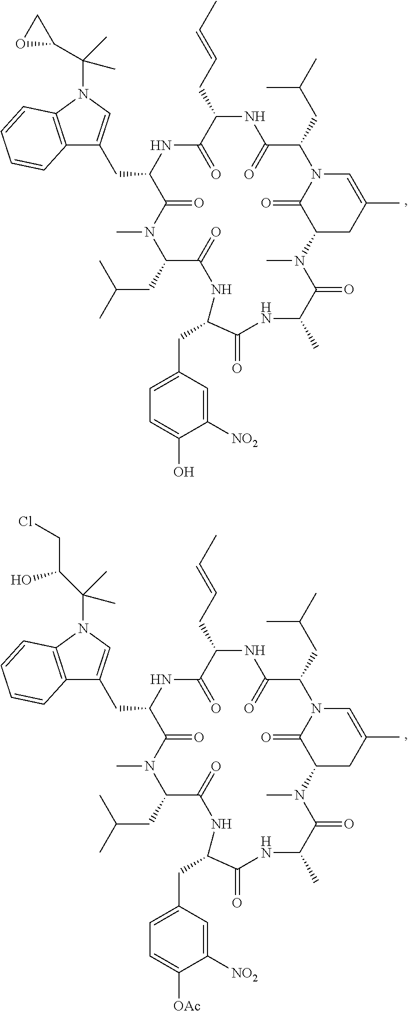 [SMILES: C/C=C/C[C@@H]1NC(=O)[C@H](CC(C)C)N2C=C(C)C[C@@H](C2=O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O ; C/C=C/C[C@@H]1NC(=O)[C@H](CC(C)C)N2C=C(C)C[C@@H](C2=O)N(C)C(=O)[C@H](C)NC(=O)[C@H](Cc2ccc(OC(C)=O)c([N+](=O)[O-])c2)NC(=O)[C@H](CC(C)C)N(C)C(=O)[C@H](Cc2cn(C(C)(C)[C@H](O)CCl)c3ccccc23)NC1=O]

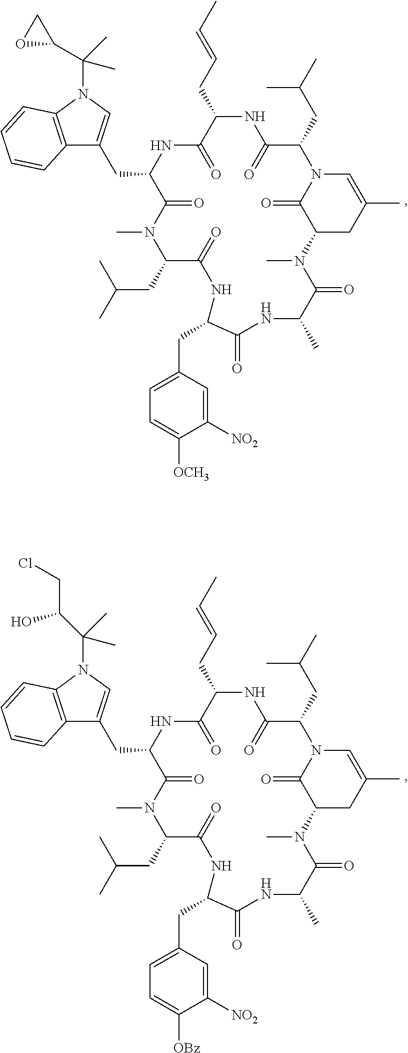

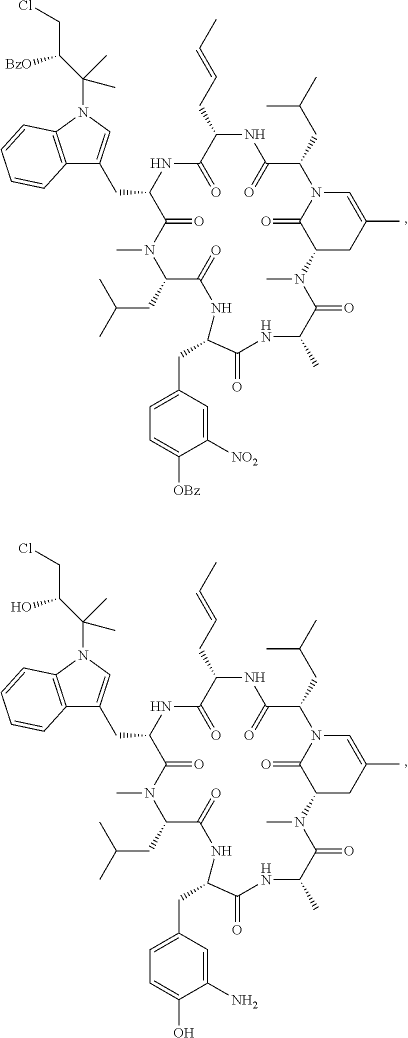

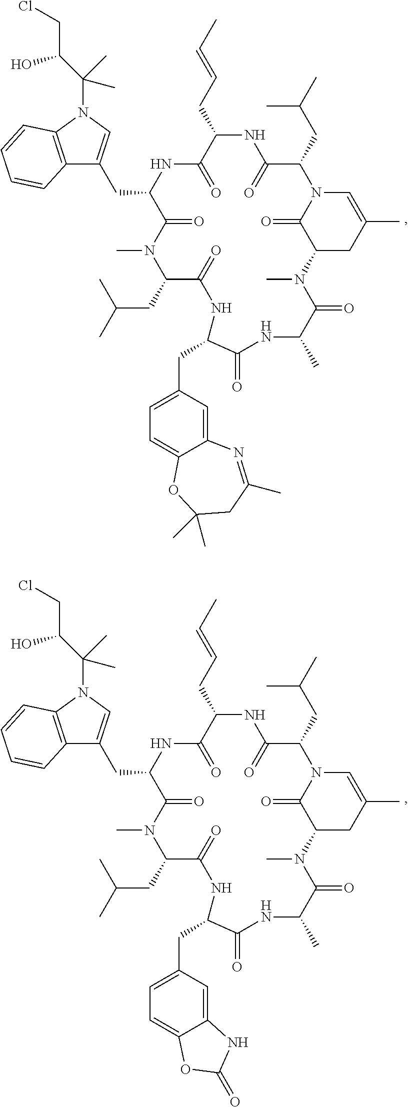 [SMILES: C/C=C/C[C@@H]1NC(=O)[C@H](CC(C)C)N2C=C(C)C[C@@H](C2=O)N(C)C(=O)[C@H](C)NC(=O)[C@H](Cc2ccc3c(c2)N=C(C)CC(C)(C)O3)NC(=O)[C@H](CC(C)C)N(C)C(=O)[C@H](Cc2cn(C(C)(C)[C@H](O)CCl)c3ccccc23)NC1=O ; C/C=C/C[C@@H]1NC(=O)[C@H](CC(C)C)N2C=C(C)C[C@@H](C2=O)N(C)C(=O)[C@H](C)NC(=O)[C@H](Cc2ccc3oc(=O)[nH]c3c2)NC(=O)[C@H](CC(C)C)N(C)C(=O)[C@H](Cc2cn(C(C)(C)[C@H](O)CCl)c3ccccc23)NC1=O]

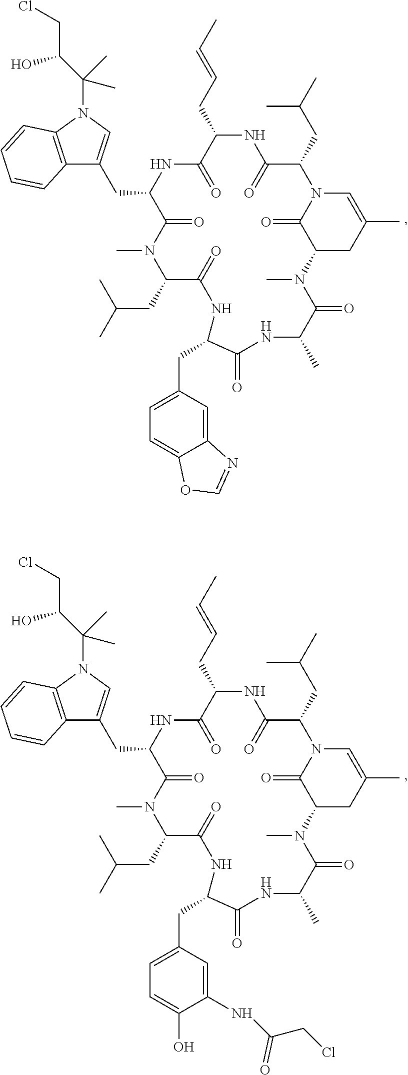 [SMILES: C/C=C/C[C@@H]1NC(=O)[C@H](CC(C)C)N2C=C(C)C[C@@H](C2=O)N(C)C(=O)[C@H](C)NC(=O)[C@H](Cc2ccc3ocnc3c2)NC(=O)[C@H](CC(C)C)N(C)C(=O)[C@H](Cc2cn(C(C)(C)[C@H](O)CCl)c3ccccc23)NC1=O ; C/C=C/C[C@@H]1NC(=O)[C@H](CC(C)C)N2C=C(C)C[C@@H](C2=O)N(C)C(=O)[C@H](C)NC(=O)[C@H](Cc2ccc(O)c(NC(=O)CCl)c2)NC(=O)[C@H](CC(C)C)N(C)C(=O)[C@H](Cc2cn(C(C)(C)[C@H](O)CCl)c3ccccc23)NC1=O]

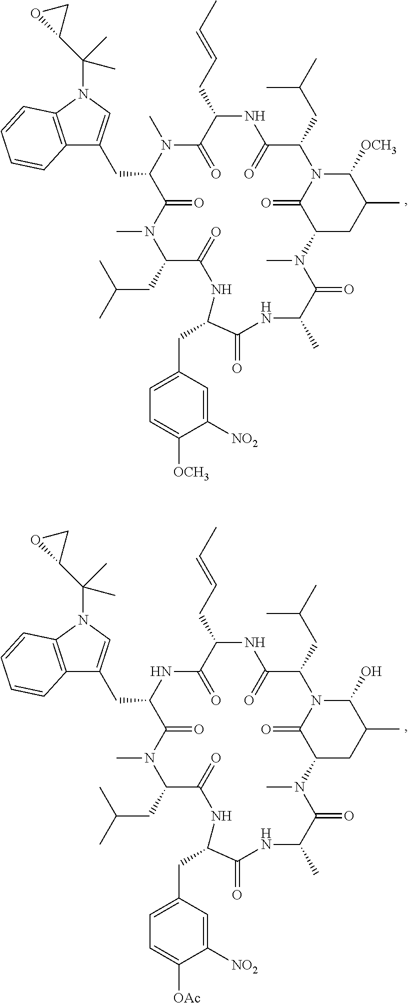 [SMILES: C/C=C/C[C@@H]1NC(=O)[C@H](CC(C)C)N2C(=O)[C@H](CC(C)[C@H]2OC)N(C)C(=O)[C@H](C)NC(=O)[C@H](Cc2ccc(OC)c([N+](=O)[O-])c2)NC(=O)[C@H](CC(C)C)N(C)C(=O)[C@H](Cc2cn(C(C)(C)[C@H]3CO3)c3ccccc23)N(C)C1=O ; C/C=C/C[C@@H]1NC(=O)[C@H](CC(C)C)N2C(=O)[C@H](CC(C)[C@H]2O)N(C)C(=O)[C@H](C)NC(=O)[C@H](Cc2ccc(OC(C)=O)c([N+](=O)[O-])c2)NC(=O)[C@H](CC(C)C)N(C)C(=O)[C@H](Cc2cn(C(C)(C)[C@H]3CO3)c3ccccc23)NC1=O]

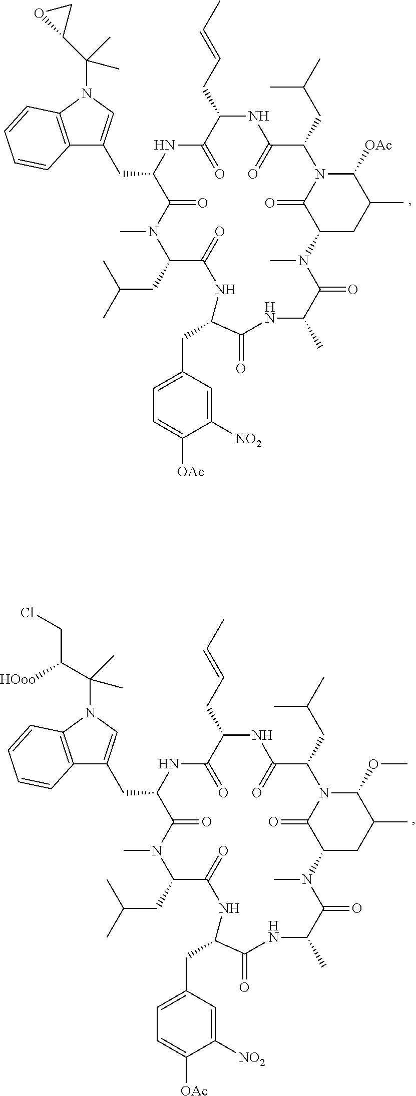

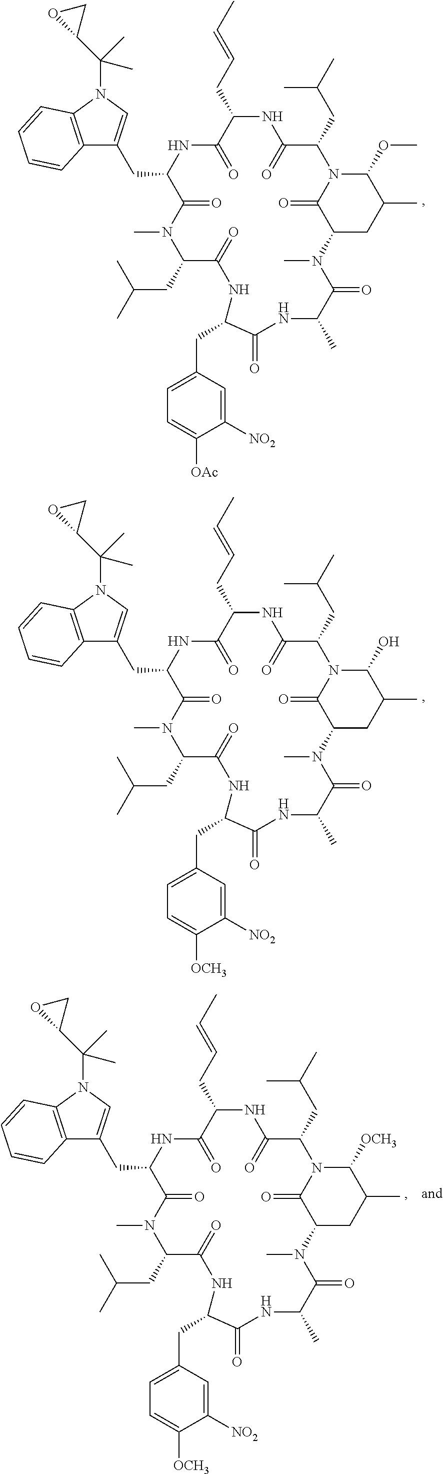

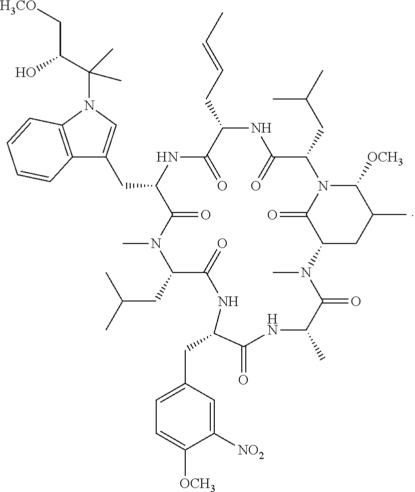 [SMILES: C/C=C/C[C@@H]1NC(=O)[C@H](CC(C)C)N2C(=O)[C@H](CC(C)[C@H]2OC)N(C)C(=O)[C@H](C)NC(=O)[C@H](Cc2ccc(OC)c([N+](=O)[O-])c2)NC(=O)[C@H](CC(C)C)N(C)C(=O)[C@H](Cc2cn(C(C)(C)[C@H](O)COC)c3ccccc23)NC1=O]

13. A compound or a pharmaceutically acceptable salt thereof having a structure selected from the group of

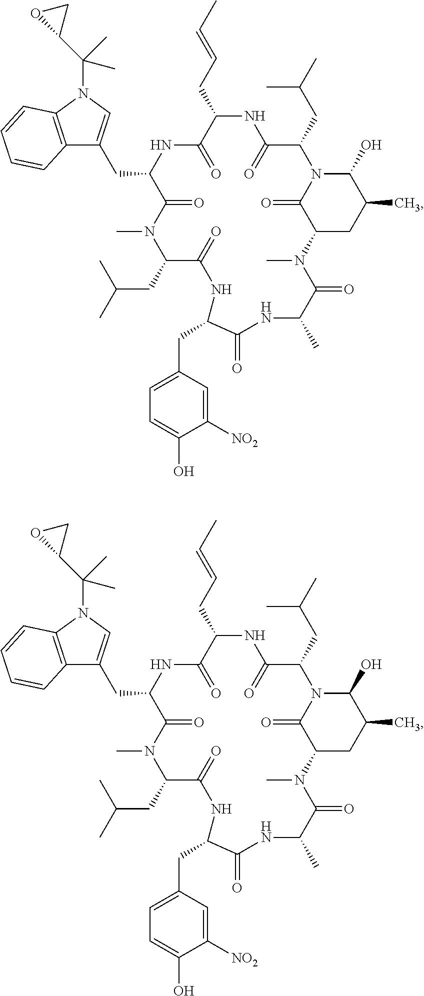 [SMILES: C/C=C/C[C@@H]1NC(=O)[C@H](CC(C)C)N2C(=O)[C@H](C[C@H](C)[C@H]2O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O ; C/C=C/C[C@@H]1NC(=O)[C@H](CC(C)C)N2C(=O)[C@H](C[C@H](C)[C@@H]2O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O]

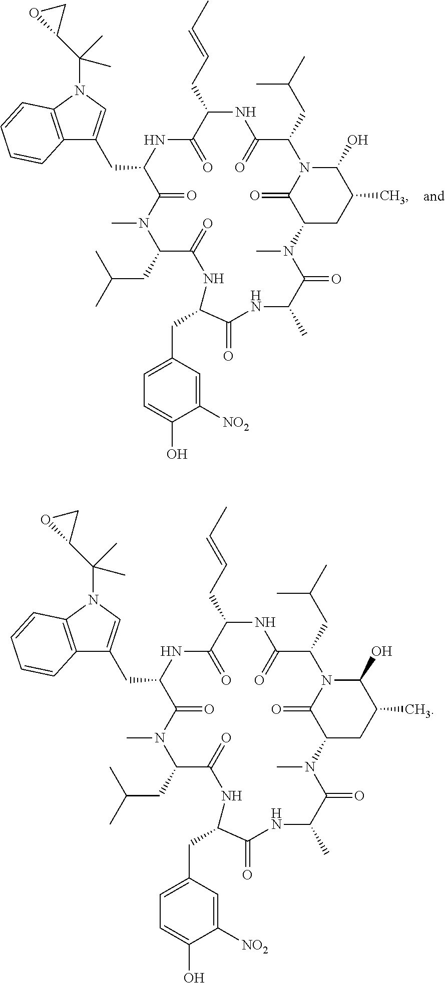 [SMILES: C/C=C/C[C@@H]1NC(=O)[C@H](CC(C)C)N2C(=O)[C@H](C[C@@H](C)[C@H]2O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O ; C/C=C/C[C@@H]1NC(=O)[C@H](CC(C)C)N2C(=O)[C@H](C[C@@H](C)[C@@H]2O)N(C)C(=O)[C@H](C)NC(=O)[C@H](Cc2ccc(O)c([N+](=O)[O-])c2)NC(=O)[C@H](CC(C)C)N(C)C(=O)[C@H](Cc2cn(C(C)(C)[C@H]3CO3)c3ccccc23)NC1=O]

14. A method of treating a bacterial infection or prophylaxis of a bacterial infection, comprising administering to a subject in need thereof a therapeutically effective amount of a compound according to claim 1, or a pharmaceutically acceptable salt thereof.

15. The method of claim 14, wherein the bacterial infection is due to Mycobacterium tuberculosis.

16. The method of claim 15, wherein the Mycobacterium tuberculosis is a drug resistant Mycobacterial strain.

17. A pharmaceutical composition comprising a therapeutically effective amount of the compound of claim 1, or a pharmaceutically acceptable salt thereof, and an excipient.

18. A method of treating a bacterial infection or prophylaxis of a bacterial infection, comprising administering to a subject in need thereof a therapeutically effective amount of a compound according to claim 13, or a pharmaceutically acceptable salt thereof.

19. The method of claim 18, wherein the bacterial infection is due to Mycobacterium tuberculosis.

20. The method of claim 19, wherein the Mycobacterium tuberculosis is a drug resistant Mycobacterial strain.
